(19) United States
(12) Patent Application Publication
(10) Pub. No.: US 2026/0253692 A1
(43) Pub. Date: Aug. 27, 2026

(54) SYSTEM

(71) Applicant: SoftBank Group Corp., Tokyo (JP)

(72) Inventor: Sayaka KUBOUCHI, Tokyo (JP)

(73) Assignee: SoftBank Group Corp., Tokyo (JP)

(21) Appl. No.: 19/542,668

(22) Filed: Feb. 18, 2026

(30) Foreign Application Priority Data
Feb. 21, 2025 (JP) 2025-026985

(51) Int. Cl. G16H 10/60; G01C 21/34; G06F 16/29; G06F 40/284; G06T 7/00; G06V 10/764; G06V 10/82; G06V 40/16; G10L 25/24; G10L 25/63; G10L 25/66

(52) U.S. Cl. CPC G16H 10/60; G01C 21/3446; G01C 21/3484; G01C 21/3492; G06F 16/29; G06T 7/0012; G06V 10/764; G06V 10/82; G06V 40/174; G10L 25/24; G10L 25/63; G10L 25/66; G06F 40/284

(57) ABSTRACT

The system according to the embodiment comprises a reception unit, an analysis unit, a provision unit, and a decision unit. The reception unit receives input of the user's symptoms and conditions. The analysis unit analyzes information received by the reception unit and determines the user's symptoms and conditions. The provision unit provides information determined by the analysis unit to an emergency call receiving agency or a medical institution. The decision unit determines deployment of emergency vehicles or a transport route based on the information provided by the provision unit.

### CROSS-REFERENCE TO RELATED APPLICATION

The present application claims priority to and incorporates by reference the entire contents of Japanese Patent Application No. 2025-026985 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that emergency call systems have difficulty accurately determining a user's symptoms and conditions and responding promptly.

### SUMMARY OF THE INVENTION

The system according to the embodiment comprises a reception unit, an analysis unit, a provision unit, and a decision unit. The reception unit receives input of the user's symptoms and conditions. The analysis unit analyzes information received by the reception unit and determines the user's symptoms and conditions. The provision unit provides information determined by the analysis unit to an emergency call receiving agency or a medical institution. The decision unit determines deployment of emergency vehicles or a transport route based on the information provided by the provision unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

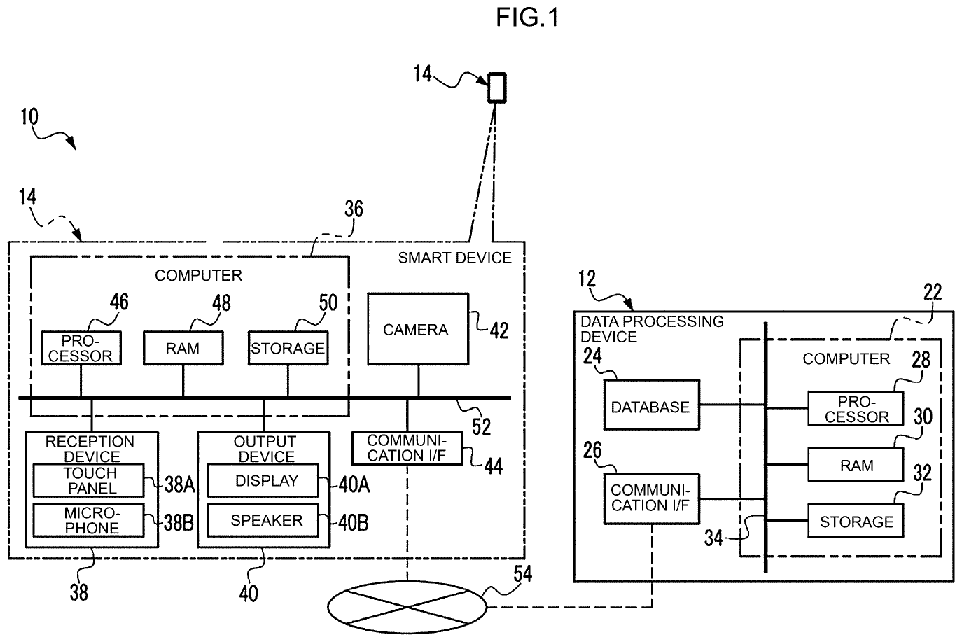

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

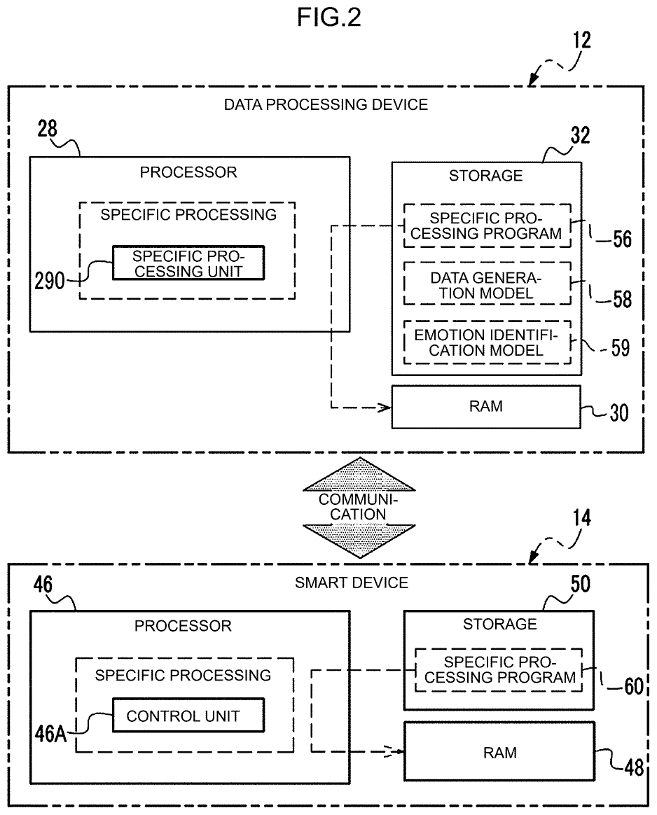

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The emergency call system according to the embodiment of the present invention is a system that utilizes devices such as smartphones and generative AI to accurately determine a user's symptoms and conditions and promptly provide necessary rescue. When a user faces an emergency, the emergency call system allows the user to input symptoms and conditions via a smartphone, and generative AI analyzes this information to make accurate judgments and arrange appropriate rescue. The generative AI rapidly provides advanced information to emergency call receiving agencies and medical institutions, and determines the deployment of emergency vehicles and optimal transport routes. This system streamlines the emergency call mechanism, enabling prompt medical care for those truly in need. For example, when a user inputs symptoms and conditions using a smartphone, the generative AI analyzes the information and arranges appropriate rescue. Furthermore, the generative AI provides advanced information to emergency call receiving agencies and medical institutions, and determines the deployment of emergency vehicles and optimal transport routes. Additionally, the generative AI continuously monitors the user's symptoms and conditions and provides additional information as needed. As a result, more precious lives can be saved. Thus, the emergency call system can accurately determine the user's symptoms and conditions and promptly provide necessary rescue. Specifically, the emergency call system enables users to input voice, text, images, and vital data (e.g., heart rate, blood pressure, blood oxygen saturation, etc.) via user interfaces such as smartphones and wearable devices. The system preprocesses these diverse input data as, for example, time-series tensors (e.g., one-dimensional arrays representing heart rate changes over 30 seconds), image tensors (e.g., photos of complexion or injury sites), and text vectors (e.g., free-form descriptions of symptoms), performing normalization, noise removal, and feature extraction (e.g., emotion estimation from voice, trauma detection from images). As generative AI, large language models (LLMs) or multimodal transformers (Transformer-based models) are used to integratively analyze the input data. Examples of input include text such as “chest pain,” “fell and hit my head,” “rapid breathing,” time-series data of heart rate at 120 bpm, and images showing pale complexion. The generative AI receives these inputs and generates outputs such as severity scores for symptoms (e.g., a score from 0 to 1, with 0.85 indicating severe), emergency labels (e.g., immediate transport, observation only), and recommended rescue measures (e.g., ambulance request, remote diagnosis). Example outputs include “severity 0.92, emergency: immediate transport, recommendation: ambulance request” and “severity 0.35, emergency: observation, recommendation: rest at home.” These outputs are used in subsequent processing such as threshold judgment (e.g., ambulance request for severity above 0.7), rule-based branching (e.g., immediate transport for trauma plus consciousness disorder), and are utilized for automatic notification to emergency call receiving agencies or medical institutions, automatic deployment instructions for emergency vehicles, and automatic calculation of optimal transport routes (e.g., shortest path search algorithms linked with geographic information systems). Furthermore, the system continuously monitors the user's condition, analyzes changes in vital data and location information in real time, and automatically sends additional information (e.g., latest heart rate, location coordinates, consciousness level) to medical institutions when symptoms worsen. As a technical effect, the present invention achieves faster decision-making, improved accuracy, reduced false reports, and optimized communication load by combining AI-based integrated analysis of high-dimensional data and rule-based processing, without relying on human operators for hearing and judgment. Application fields include urban emergency medical care, remote medical support in depopulated areas, mass notification systems during disasters, and employee health management in companies. Thus, the emergency call system contributes not only to the automation of human tasks but also to the improvement of computer technology itself and the advancement of social infrastructure.

The emergency call system according to the embodiment comprises a reception unit, an analysis unit, a provision unit, and a decision unit. The reception unit receives input of the user's symptoms and conditions. The user's symptoms and conditions may include, for example, health status, emergencies, accidents, and the like, but are not limited thereto. The reception unit can receive input from the user, for example, via a smartphone application. The reception unit may also support multiple input methods, such as voice input and text input. The analysis unit uses generative AI to analyze information received by the reception unit and determine the user's symptoms and conditions. The analysis may be performed based on data analysis algorithms or diagnostic criteria, but is not limited thereto. For example, the generative AI analyzes the user's input information and determines the severity and urgency of symptoms. The analysis unit may also use generative AI to continuously monitor the user's symptoms and conditions and provide additional information as needed. The provision unit uses generative AI to provide information determined by the analysis unit to emergency call receiving agencies or medical institutions. The provision may be performed based on communication means or information formats, but is not limited thereto. For example, the provision unit transmits detailed information about the user's symptoms and conditions to emergency call receiving agencies or medical institutions. The decision unit uses generative AI to determine the deployment of emergency vehicles or transport routes based on information provided by the provision unit. The decision may be performed based on urgency evaluation criteria or route selection algorithms, but is not limited thereto. For example, the decision unit determines the deployment of emergency vehicles or optimal transport routes and arranges prompt rescue. Thus, the emergency call system according to the embodiment can accurately determine the user's symptoms and conditions and promptly provide necessary rescue. Specifically, the emergency call system is designed so that the reception unit uses applications on smartphones or wearable devices, allowing users to input voice, text, images, and vital data (e.g., heart rate, blood pressure, blood oxygen saturation, etc.). For voice input, the reception unit samples the voice signal, performs spectrogram conversion and noise removal, and then converts it to text using a speech recognition model (e.g., Transformer-based speech recognition network). For text input, preprocessing for natural language processing (e.g., tokenization, stop word removal) is performed and vectorized. For image input, the image is normalized as an image tensor (e.g., 224×224×3 RGB image), and feature extraction is performed using a convolutional neural network (CNN). Vital data is handled as a time-series tensor (e.g., a one-dimensional array representing heart rate changes over 60 seconds) and analyzed using a recurrent neural network (RNN) or time-series transformer. The analysis unit integrates these diverse input data and uses a multimodal transformer (e.g., Transformer handling images, voice, and text simultaneously) to output severity scores for symptoms (e.g., a score from 0 to 1), emergency labels (e.g., immediate transport, observation only), and recommended rescue measures (e.g., ambulance request, remote diagnosis). Examples of input include text such as “chest pain,” “fell and hit my head,” “rapid breathing,” time-series data of heart rate at 120 bpm, and images showing pale complexion. The analysis unit receives these inputs and generates outputs such as severity 0.92, emergency: immediate transport, recommendation: ambulance request. The outputs are used in subsequent processing such as threshold judgment (e.g., ambulance request for severity above 0.7), rule-based branching (e.g., immediate transport for trauma plus consciousness disorder). The provision unit automatically transmits the output of the analysis unit as structured data (e.g., JSON format) to emergency call receiving agencies or medical institutions, and uses communication protocols (e.g., HTTPS, MQTT) to transmit information in real time. The decision unit uses the received information and, in cooperation with geographic information systems (GIS), applies shortest path search algorithms (e.g., Dijkstra's algorithm, A* algorithm) to determine the deployment of emergency vehicles or transport routes. Furthermore, the decision unit refers to past transport history and current traffic condition data (e.g., real-time congestion information from traffic APIs) to automatically generate optimal deployment and transport plans. As a technical effect, the system achieves faster decision-making, improved accuracy, reduced false reports, and optimized communication load by combining AI-based integrated analysis of high-dimensional data and rule-based processing, without relying on human operators for hearing and judgment. Application fields include urban emergency medical care, remote medical support in depopulated areas, mass notification systems during disasters, and employee health management in companies. Thus, the emergency call system contributes not only to the automation of human tasks but also to the improvement of computer technology itself and the advancement of social infrastructure.

The emergency call system comprises a monitoring unit configured to continuously monitor the user's symptoms and conditions. The monitoring unit continuously monitors the user's symptoms and conditions. Monitoring may be performed by methods such as real-time monitoring or periodic checks, but is not limited thereto. For example, the monitoring unit monitors the user's health status in real time and issues an alert when an abnormality is detected. The monitoring unit may also periodically check the user's health status to confirm the absence of abnormalities. By continuously monitoring the user's symptoms and conditions, necessary rescue can be promptly provided. Specifically, the monitoring unit periodically or in real time acquires the user's vital data (e.g., heart rate, blood pressure, blood oxygen saturation, body temperature, etc.) and behavioral data (e.g., step count, fall detection, sleep duration) from sensors in wearable devices or smartphones. The acquired data is recorded as time-series tensors (e.g., one-dimensional arrays representing heart rate changes per minute) or event logs (e.g., timestamped records of fall events). The monitoring unit preprocesses these data (e.g., noise removal, outlier correction, normalization) and applies anomaly detection algorithms (e.g., reconstruction error judgment by autoencoders, time-series anomaly scoring by LSTM). Examples of input include 60 seconds of heart rate data ([80, 82, 85, 120, 130, 125, . . . ]), and the occurrence time of a fall event (2024-06-01 14:23:10). Outputs of anomaly detection include anomaly scores (e.g., 0.95), anomaly labels (e.g., fall detection), and alert flags (e.g., True), and threshold judgment is performed, such as issuing an alert when the anomaly score exceeds 0.8. When an alert is generated, the monitoring unit automatically sends anomaly information to the analysis unit or provision unit, enabling prompt rescue arrangements and notification to medical institutions. Furthermore, when the monitoring unit periodically checks the user's health status, it generates daily or weekly summary reports, performs trend analysis of health status (e.g., transitions in average and standard deviation of heart rate), and risk prediction (e.g., upward trend in anomaly scores over the past week). As a technical effect, the monitoring unit achieves high-frequency, high-precision anomaly detection using sensor data and AI, without relying on intermittent human observation or self-reporting, thereby preventing rescue delays and significantly reducing false reports and missed detections. Application fields include monitoring of elderly persons, remote health management for chronic disease patients, employee health monitoring in companies, and mass health surveillance during disasters. Thus, the emergency call system contributes not only to the automation of human tasks but also to the improvement of computer technology itself and the enhancement of social safety.

The monitoring unit comprises an additional provision unit configured to provide additional information when the user's symptoms worsen. The additional provision unit provides additional information when the user's symptoms worsen. Additional information may include, for example, details of symptoms or changes in urgency, but is not limited thereto. For example, the additional provision unit provides detailed information about symptoms to emergency call receiving agencies or medical institutions when the user's symptoms worsen. The additional provision unit may also provide information when the urgency of the user's symptoms changes. By providing additional information when the user's symptoms worsen, prompt response becomes possible. Specifically, the additional provision unit executes additional information generation processing triggered by anomaly detection results received from the monitoring unit (e.g., sudden increase in heart rate, fall detection, decrease in consciousness level). Additional information includes recent time-series vital data (e.g., heart rate and blood pressure trends over the past 10 minutes), latest location information (e.g., GPS coordinates), user's medical history and medication information, and recent behavioral history (e.g., walking pattern before a fall). These pieces of information are compiled as structured data (e.g., JSON format) and automatically sent to emergency call receiving agencies or medical institutions via the provision unit. Examples of input include heart rate data [80, 82, 120, 130], GPS coordinates (35.6895, 139.6917), and medical history “hypertension.” The additional provision unit uses generative AI (e.g., large language models or multimodal transformers) to analyze patterns of symptom worsening and outputs urgency scores (e.g., 0.95) and recommended responses (e.g., immediate transport). The outputs are used in subsequent processing such as threshold judgment (e.g., immediate notification for urgency above 0.8), rule-based branching (e.g., ambulance request for fall plus consciousness disorder). As a technical effect, the additional provision unit eliminates the need for sequential hearing by human operators and achieves automatic and comprehensive additional information generation and immediate notification by AI, thereby preventing rescue delays and misjudgments due to lack of information. Application fields include monitoring of elderly persons, home medical care, remote health management, and mass health surveillance during disasters. Thus, the additional provision unit contributes not only to the automation of human tasks but also to the improvement of computer technology itself and the advancement of medical and rescue activities.

The analysis unit is capable of analyzing the user's symptoms and conditions and arranging appropriate rescue. The analysis unit uses generative AI to analyze the user's symptoms and conditions and arrange appropriate rescue. Appropriate rescue may include, for example, arranging an ambulance or contacting a medical institution, but is not limited thereto. For example, the analysis unit analyzes the user's symptoms and arranges an ambulance. The analysis unit may also analyze the user's conditions and contact a medical institution. By analyzing the user's symptoms and conditions and arranging appropriate rescue, prompt response becomes possible. Specifically, the analysis unit integratively preprocesses diverse input data received from the reception unit (e.g., voice, text, images, vital data), performing spectrogram conversion and speech recognition for voice, vectorization by natural language processing for text, feature extraction by CNN for images, and time-series analysis (e.g., LSTM or autoregressive models) for vital data. These features are input to a multimodal transformer (e.g., Transformer-based integrated model), which outputs severity scores for symptoms (e.g., a score from 0 to 1), emergency labels (e.g., immediate transport, observation only), and recommended rescue measures (e.g., ambulance request, remote diagnosis). Examples of input include text such as “chest pain,” “fell and hit my head,” time-series data of heart rate at 120 bpm, and images showing pale complexion. The analysis unit receives these inputs and generates outputs such as severity 0.92, emergency: immediate transport, recommendation: ambulance request, and the outputs are used in subsequent processing such as threshold judgment (e.g., ambulance request for severity above 0.7), rule-based branching (e.g., immediate transport for trauma plus consciousness disorder). Furthermore, the analysis unit continuously monitors the user's condition, analyzes changes in vital data and location information in real time, and automatically sends additional information (e.g., latest heart rate, location coordinates, consciousness level) to medical institutions when symptoms worsen. As a technical effect, the analysis unit achieves faster decision-making, improved accuracy, reduced false reports, and optimized communication load by combining AI-based integrated analysis of high-dimensional data and rule-based processing, without relying on human operators for hearing and judgment. Application fields include urban emergency medical care, remote medical support in depopulated areas, mass notification systems during disasters, and employee health management in companies. Thus, the analysis unit contributes not only to the automation of human tasks but also to the improvement of computer technology itself and the advancement of social infrastructure.

The provision unit is capable of providing detailed information to an emergency call receiving agency or a medical institution. The provision unit uses generative AI to provide detailed information to emergency call receiving agencies or medical institutions. Detailed information may include, for example, the patient's medical history or current symptoms, but is not limited thereto. For example, the provision unit provides the patient's medical history to an emergency call receiving agency. The provision unit may also provide detailed information about current symptoms to a medical institution. By providing advanced information to emergency call receiving agencies or medical institutions, prompt response becomes possible. Specifically, the provision unit integrates severity scores for symptoms, emergency labels, recommended rescue measures, time-series vital data, image features, medical history, medication information, and latest location information received from the analysis unit, and automatically sends them as structured data (e.g., JSON format) to emergency call receiving agencies or medical institutions. During transmission, communication protocols (e.g., HTTPS, MQTT) are used to transmit information in real time, and encryption and authentication processing are also performed. Examples of input include severity 0.92, emergency: immediate transport, recommendation: ambulance request, heart rate data [120, 130, 125], medical history “hypertension,” and location information (35.6895, 139.6917). The provision unit performs format conversion of information (e.g., medical standard formats such as HL7, FHIR) and prioritization (e.g., priority transmission based on emergency labels) according to the requirements of the receiving system. Furthermore, the provision unit is equipped with functions for delivery confirmation after transmission, retransmission control, and automatic retry in case of communication failure. As a technical effect, the provision unit eliminates the need for manual input or telephone contact by human operators and achieves automatic and comprehensive information generation and immediate notification by AI, thereby preventing rescue delays and information transmission errors. Application fields include emergency medical sites, remote medical support, mass notification during disasters, and corporate health management systems. Thus, the provision unit contributes not only to the automation of human tasks but also to the improvement of computer technology itself and the advancement of medical and rescue activities.

The decision unit is capable of determining the deployment of emergency vehicles or an optimal transport route. The decision unit uses generative AI to determine the deployment of emergency vehicles or optimal transport routes. The decision may be performed based on urgency evaluation criteria or route selection algorithms, but is not limited thereto. For example, the decision unit determines the deployment of emergency vehicles based on urgency evaluation criteria. The decision unit may also determine the optimal transport route based on route selection algorithms. By determining the deployment of emergency vehicles or optimal transport routes, prompt response becomes possible. Specifically, the decision unit integrates severity scores for symptoms, emergency labels, location information, traffic condition data (e.g., real-time congestion information, accident/construction information), and past transport history received from the provision unit, and uses shortest path search algorithms (e.g., Dijkstra's algorithm, A* algorithm) in cooperation with geographic information systems (GIS) to determine the deployment of emergency vehicles or transport routes. Examples of input include emergency: immediate transport, location information (35.6895, 139.6917), traffic congestion information “congestion at point A,” and past transport history “success rate of transport to hospital B: 95%.” When the urgency score is high, the decision unit immediately deploys the nearest emergency vehicle and automatically selects the shortest and fastest transport route according to traffic conditions. Outputs include deployment instructions (e.g., vehicle ID, dispatch time), transport routes (e.g., list of waypoints, estimated arrival time), and priority labels (e.g., highest priority), which are automatically sent to emergency vehicle operation systems or medical institutions. Furthermore, the decision unit monitors changes in traffic conditions during transport in real time and recalculates routes or changes deployment as needed. As a technical effect, the decision unit eliminates the need for manual judgment or telephone contact by human operators and achieves automatic and optimized deployment and transport plan generation by AI, thereby preventing rescue delays and misdeployment. Application fields include urban emergency medical care, remote medical support in depopulated areas, mass transport during disasters, and employee health management in companies. Thus, the decision unit contributes not only to the automation of human tasks but also to the improvement of computer technology itself and the advancement of medical and rescue activities.

The reception unit is capable of estimating the user's emotions and adjusting the input method for symptoms and conditions based on the estimated emotions. The reception unit uses generative AI to estimate the user's emotions and adjust the input method for symptoms and conditions based on the estimated emotions. For example, if the user is in a panic state, the reception unit provides a simple and intuitive interface and minimizes the input procedure. If the user is calm, the reception unit provides detailed input options and proposes customizable input methods. Furthermore, if the user is facing an emergency, the reception unit prioritizes voice input to enable rapid input of symptoms and conditions. By adjusting the input method according to the user's emotions, more appropriate information can be entered quickly. Emotion estimation is realized using emotion engines or generative AI with emotion estimation functions. Generative AI may be a text generative AI (e.g., LLM) or a multimodal generative AI, but is not limited thereto. Specifically, the reception unit receives as input voice data obtained from the user (e.g., 1-minute voice waveform sampled at 16 kHz), text data (e.g., natural language sentences such as “Please help, I can't breathe”), image data (e.g., facial photos or expression images), and vital data (e.g., heart rate, skin conductance, etc.). The reception unit performs spectrogram conversion and acoustic feature extraction (e.g., MFCC, pitch, energy) on voice data, and expression feature extraction using convolutional neural networks (CNN) on image data. Text data is processed by a natural language processing model (e.g., large language model) to estimate emotion labels (e.g., anger, anxiety, fear, calmness) and emotion scores (e.g., 0.85 for “panic”). Vital data is analyzed by time-series analysis models (e.g., LSTM) to calculate stress indices and abnormal values. These diverse features are input to a multimodal transformer (Transformer-based integrated model), which outputs the final emotion estimation result (e.g., emotion category “panic,” confidence 0.92). Example outputs include “emotion: panic, confidence 0.92” and “emotion: calm, confidence 0.75.” Based on this emotion estimation result, the reception unit dynamically changes the configuration of the user interface. For example, in a panic state, a simple UI is displayed that allows emergency requests with a single button, and voice input is automatically activated. In a calm state, detailed symptom selection options and free description fields are displayed, allowing the user to select customizable input methods. The emotion estimation result is passed to subsequent input guide modules and input content filtering modules, and is used for simplification or detailing of input procedures and automatic selection of input items. As a technical effect, the reception unit responds immediately to psychological changes in users during emergencies, which were difficult to handle with conventional uniform input interfaces, and achieves significant reduction of input errors and delays and rapid and accurate emergency calls by combining high-precision emotion estimation by AI and automatic interface optimization. Application fields include emergency medical call apps, mass notification systems during disasters, elderly monitoring terminals, and corporate safety management systems. Thus, the reception unit achieves improvement of computer technology itself by dynamically optimizing computer interfaces according to user status, beyond mere automation of human tasks.

The reception unit is capable of analyzing the user's past emergency call history and providing an appropriate input interface. The reception unit uses generative AI to analyze the user's past emergency call history and provide an optimal input interface. For example, the reception unit preferentially proposes input methods (voice, text, etc.) that the user has frequently used in the past. The reception unit may also automatically display input options corresponding to specific symptoms or conditions based on the user's past emergency call history. Furthermore, the reception unit may analyze the user's past emergency call history and propose input interfaces used during specific time periods. By analyzing the user's past emergency call history, the reception unit can provide an optimal input interface. Specifically, the reception unit maintains a database of past emergency call history for each user (e.g., structured data including time, input method, symptom category, input duration, success rate, etc.) and inputs this to generative AI. Examples of input include “2024-06-01 14:23:10, voice input, chest pain, input duration 15 seconds,” “2024-05-20 08:10:00, text input, fall, input duration 30 seconds,” etc. The reception unit preprocesses these history data as time-series vectors or categorical features and applies algorithms such as frequency analysis (e.g., 8 out of 10 past cases used voice input), clustering (e.g., input tendencies by symptom), and time pattern extraction (e.g., voice input is more common at night). Generative AI (e.g., large language models or time-series analysis models) uses these features to recommend optimal input interfaces for each user (e.g., prioritize voice input, prioritize text input, combine image input) and input options (e.g., frequently used symptom templates, auto-completion candidates from past input history). Example outputs include “recommended input method: voice input, recommended symptom options: chest pain, fall,” “recommended time period: text input in the morning,” etc. These outputs are passed to the UI generation module of the reception unit, and customized input screens and procedures are automatically generated for each user. As a technical effect, the reception unit achieves user-specific optimization, which was difficult to realize with conventional uniform input interfaces, by combining AI-based history analysis and automatic interface generation, greatly improving input efficiency, accuracy, and user satisfaction. Application fields include personalized emergency call apps, regular reporting systems for chronic disease patients, and corporate safety reporting platforms. Thus, the reception unit achieves advancement of computer technology by optimizing interfaces based on user history, beyond mere automation of human tasks.

The reception unit is capable of filtering input content for symptoms and conditions based on the user's current health status and medical history at the time of input. The reception unit uses generative AI to filter input content for symptoms and conditions based on the user's current health status and medical history at the time of input. For example, the reception unit prioritizes input of relevant symptoms and conditions based on the user's medical history. The reception unit may also automatically filter input content based on the user's current health status and highlight important information. Furthermore, the reception unit may refer to the user's health data and perform filtering to optimize input content. By filtering input content based on the user's current health status and medical history, important information can be highlighted. Specifically, the reception unit obtains the user's electronic health records (EHR) and vital database (e.g., medical history “hypertension,” “diabetes,” current heart rate 120 bpm, blood pressure 150/95 mmHg, etc.) as input. These data are input to generative AI as structured data vectors (e.g., disease category, medication history, recent vital values). Generative AI (e.g., large language models or rule-based filtering modules) outputs a prioritized list of candidate symptoms and conditions for input based on combinations of medical history and current values. Examples of input include “medical history: hypertension, current: headache, blood pressure 150/95,” “medical history: asthma, current: dyspnea,” etc. Example outputs include “priority input candidates: headache, chest pain, dizziness,” “important highlighted items: dyspnea, consciousness disorder,” etc. The reception unit uses these outputs to highlight important items on the input screen and hide unnecessary items. Furthermore, real-time feedback for auto-completion of input content and prevention of input errors can also be implemented. As a technical effect, the reception unit achieves individual optimization, which was difficult with conventional uniform input item presentation, by combining AI-based health data analysis and input content filtering, greatly improving input efficiency, accuracy, and information transmission accuracy in medical settings. Application fields include emergency call apps, self-reporting systems for chronic disease patients, and remote medical platforms. Thus, the reception unit achieves improvement of computer technology by optimizing input through health data linkage, beyond mere automation of human tasks.

The reception unit is capable of estimating the user's emotions and determining the priority of input content based on the estimated emotions. The reception unit uses generative AI to estimate the user's emotions and determine the priority of input content based on the estimated emotions. For example, if the user is nervous, the reception unit prioritizes input of important information. If the user is relaxed, the reception unit allows input of detailed information. Furthermore, if the user is in a hurry, the reception unit enables rapid input of only the most important information. By determining the priority of input content according to the user's emotions, important information can be entered quickly. Emotion estimation is realized using emotion engines or generative AI with emotion estimation functions. Generative AI may be a text generative AI (e.g., LLM) or a multimodal generative AI, but is not limited thereto. Specifically, the reception unit receives as input voice data obtained from the user (e.g., 30 seconds of voice waveform), text data (e.g., natural language sentences such as “Help, I'm suffering”), image data (e.g., facial photos), and vital data (e.g., heart rate, skin conductance). The reception unit performs acoustic feature extraction (e.g., MFCC, pitch) on voice data, expression feature extraction using CNN on image data, and emotion classification (e.g., nervous, relaxed, hurried) using large language models on text data. Vital data is analyzed by time-series analysis models to calculate stress indices. These features are input to a multimodal transformer, which outputs emotion categories (e.g., nervous, relaxed, hurried) and confidence scores. Example outputs include “emotion: nervous, confidence 0.88,” “emotion: relaxed, confidence 0.72,” etc. Based on this emotion estimation result, the reception unit automatically determines the priority of input content. For example, in a nervous state, the most important items such as “current symptoms,” “occurrence time,” and “consciousness level” are displayed at the top, while in a relaxed state, detailed items such as “medical history” and “medication information” are entered sequentially. In a hurry, only the minimum required items are entered immediately. These priority information are passed to input guide modules and UI generation modules, and are used for dynamic configuration of input screens and automatic optimization of input procedures. As a technical effect, the reception unit responds immediately to psychological changes in users during emergencies, which were difficult to handle with conventional uniform input order, and achieves significant reduction of input delays and information omissions and rapid and accurate emergency calls by combining AI-based emotion estimation and input priority control. Application fields include emergency medical call apps, mass notification systems during disasters, and elderly monitoring terminals. Thus, the reception unit achieves improvement of computer technology by input control according to user status, beyond mere automation of human tasks.

The reception unit is capable of prioritizing input of highly relevant information based on the user's current geographic location at the time of input of symptoms and conditions. The reception unit uses generative AI to prioritize input of highly relevant information based on the user's geographic location at the time of input of symptoms and conditions. For example, if the user is in a specific region, the reception unit prioritizes input of symptoms and conditions relevant to that region. The reception unit may also prioritize input of information about relevant medical institutions or emergency call receiving agencies based on the user's current location. Furthermore, the reception unit may refer to the user's geographic location and propose optimal input content. By considering the user's geographic location, highly relevant information can be entered preferentially. Specifically, the reception unit receives latitude and longitude data (e.g., 35.6895, 139.6917) obtained from the GPS sensor of the user terminal or network location information API. The reception unit cooperates with a geographic information system (GIS) database to obtain external data such as lists of nearby medical institutions, information on regional infectious disease outbreaks, and disaster occurrence status. Generative AI (e.g., large language models or geographic information linkage modules) combines location information and external data to output prioritized lists of candidate symptoms and conditions for input (e.g., heatstroke risk, infectious disease outbreak, disaster situation) and information about the nearest medical institutions or emergency call receiving agencies. Examples of input include “location: 35.6895, 139.6917, disaster: earthquake occurred,” “location: 34.6937, 135.5023, infectious disease outbreak: influenza,” etc. Example outputs include “priority input candidates: trauma, dyspnea,” “nearest medical institution: Hospital A, Clinic B,” etc. The reception unit uses these outputs to highlight region-specific symptoms and information about the nearest institutions on the input screen and hide unnecessary items. Furthermore, auto-completion of input content and region-specific alert messages can also be displayed. As a technical effect, the reception unit achieves immediate response to regional characteristics, which was difficult with conventional uniform input item presentation, by combining AI-based location information analysis and input content optimization, greatly improving input efficiency, accuracy, and regional medical cooperation. Application fields include emergency call apps, mass notification systems during disasters, and health management apps for travelers. Thus, the reception unit achieves improvement of computer technology by optimizing input through geographic information linkage, beyond mere automation of human tasks.

The reception unit is capable of analyzing the user's social media activity and prompting input of relevant information at the time of input of symptoms and conditions. The reception unit uses generative AI to analyze the user's social media activity and prompt input of relevant information at the time of input of symptoms and conditions. For example, the reception unit extracts information related to current conditions or symptoms from the user's social media posts and proposes input content. The reception unit may also analyze the user's social media activity and prioritize input of information related to emergencies. Furthermore, the reception unit may refer to the user's social media data and propose optimal input content. By analyzing the user's social media activity, relevant information can be entered quickly. Specifically, the reception unit receives as input post data obtained from social media APIs within the scope permitted by the user (e.g., post text, images, post time, location tags from the past week). The reception unit performs natural language analysis using large language models on post text to extract keywords related to symptoms and conditions (e.g., “headache,” “fall,” “shortness of breath”) and emergency words (e.g., “help,” “ambulance”). Image data is analyzed by CNN to detect trauma or abnormal expressions, and post time and location tags are used for time-series and geographic context analysis. Generative AI integrates these features and outputs candidate symptoms and conditions for input, estimated urgency values, and prioritized input lists. Examples of input include “post: ‘I've had a headache since this morning’,” “image: pale complexion,” “post time: 2024-06-01 08:00,” etc. Example outputs include “priority input candidates: headache, fatigue,” “estimated urgency: 0.7,” etc. The reception unit uses these outputs to display related items as auto-completion candidates on the input screen, reducing the user's input burden. Furthermore, when urgency is high, the input procedure can be simplified and switched to immediate notification mode. As a technical effect, the reception unit achieves external information linkage, which was difficult with conventional user self-reporting or manual input, by combining AI-based social media analysis and input content optimization, greatly improving input efficiency, accuracy, and urgency determination accuracy. Application fields include emergency call apps, health management systems for young people, and mass notification platforms during disasters. Thus, the reception unit achieves improvement of computer technology by optimizing input through external data linkage, beyond mere automation of human tasks.

The analysis unit is capable of estimating the user's emotions and adjusting the accuracy of analysis based on the estimated emotions. The analysis unit uses generative AI to estimate the user's emotions and adjust the accuracy of analysis based on the estimated emotions. For example, if the user is nervous, the analysis unit increases the accuracy of analysis to extract important information. If the user is relaxed, the analysis unit performs detailed analysis and provides comprehensive information. Furthermore, if the user is in a hurry, the analysis unit performs rapid analysis and prioritizes important information. By adjusting the accuracy of analysis according to the user's emotions, important information can be extracted quickly. Emotion estimation is realized using emotion engines or generative AI with emotion estimation functions. Generative AI may be a text generative AI (e.g., LLM) or a multimodal generative AI, but is not limited thereto. Specifically, the analysis unit receives as input voice data obtained from the user (e.g., 30 seconds of voice waveform sampled at 16 kHz), text data (e.g., natural language sentences such as “Help, I can't breathe”), image data (e.g., facial photos or expression images), and vital data (e.g., heart rate, skin conductance, etc.). The analysis unit performs spectrogram conversion and acoustic feature extraction (e.g., MFCC, pitch, energy) on voice data, and expression feature extraction using CNN on image data. Text data is processed by a large language model to estimate emotion labels (e.g., nervous, anxious, calm) and emotion scores (e.g., 0.88 for “nervous”). Vital data is analyzed by time-series analysis models (e.g., LSTM) to calculate stress indices and abnormal values. These diverse features are input to a multimodal transformer (Transformer-based integrated model), which outputs the final emotion estimation result (e.g., emotion category “nervous,” confidence 0.88). Examples of input include “voice: trembling voice,” “text: ‘I'm suffering’,” “image: tense face,” “heart rate: 110 bpm,” etc. The analysis unit uses this emotion estimation result to dynamically change the algorithms, thresholds, feature selection, and analysis depth (e.g., whether to perform detailed time-series analysis, threshold adjustment for anomaly detection, granularity of output labels) applied at each stage of the analysis pipeline. For example, in a nervous state, thresholds are lowered to prevent false detection and important information extraction is prioritized. In a relaxed state, detailed analysis is performed and comprehensive health reports are generated. In a hurry, only the minimum required items are analyzed rapidly and important information is output immediately. Example outputs include “severity 0.92, emergency: immediate transport, recommendation: ambulance request (high-precision analysis in nervous state),” “severity 0.35, emergency: observation, recommendation: rest at home (detailed analysis in relaxed state),” etc. These outputs are passed to subsequent provision units or decision units and used to optimize rescue arrangements and notification content. As a technical effect, the analysis unit responds immediately to psychological changes in users, which were difficult to handle with conventional uniform analysis accuracy settings, and achieves simultaneous improvement of analysis accuracy, speed, and reduction of false reports by combining AI-based emotion estimation and automatic optimization of the analysis pipeline. Application fields include emergency medical call apps, mass notification systems during disasters, elderly monitoring terminals, and corporate safety management systems. Thus, the analysis unit achieves improvement of computer technology by controlling analysis accuracy according to user status, beyond mere automation of human tasks.

The analysis unit is capable of adjusting the level of detail of analysis based on the importance of symptoms and conditions during analysis. The analysis unit uses generative AI to adjust the level of detail of analysis based on the importance of symptoms and conditions during analysis. For example, in the case of serious symptoms or conditions, the analysis unit performs detailed analysis and extracts important information. In the case of minor symptoms or conditions, the analysis unit performs simplified analysis and provides information quickly. Furthermore, the analysis unit may dynamically adjust the level of detail of analysis according to the importance of symptoms and conditions. By adjusting the level of detail of analysis based on the importance of symptoms and conditions, important information can be provided quickly. Specifically, the analysis unit integratively preprocesses diverse input data received from the reception unit (e.g., voice, text, images, vital data), performing feature extraction for each data type (e.g., MFCC for voice, CNN features for images, tokenization and vectorization for text, time-series tensorization for vital data). The analysis unit performs initial judgment of symptoms and conditions (e.g., symptom classification such as “chest pain,” “fall” by large language models, vital anomaly scoring), and calculates importance scores (e.g., continuous values from 0 to 1, with 0.9 indicating serious) and emergency labels (e.g., immediate transport, observation only). Examples of input include “symptom: chest pain, heart rate 120 bpm,” “condition: fall, decreased consciousness level,” etc. When the importance score is high, the analysis unit applies a detailed analysis pipeline (e.g., multi-stage anomaly detection, cross-validation by multiple models, comparative analysis with past data), and when the score is low, only simple rule-based judgment or threshold judgment is performed. Example outputs include “detailed analysis result: severity 0.95, emergency: immediate transport, recommendation: ambulance request,” “simple analysis result: severity 0.25, emergency: observation, recommendation: rest at home,” etc. These outputs are passed to subsequent provision units or decision units and used to optimize rescue arrangements and notification content. As a technical effect, the analysis unit achieves simultaneous improvement of computational efficiency, analysis accuracy, and speed by combining AI-based importance judgment and automatic switching of the analysis pipeline, which was difficult with conventional uniform analysis detail settings, thereby optimizing resources and reducing false reports. Application fields include emergency medical call apps, mass notification systems during disasters, and corporate health management platforms. Thus, the analysis unit achieves improvement of computer technology by controlling the level of detail of analysis according to importance, beyond mere automation of human tasks.

The analysis unit is capable of applying different analysis algorithms according to the category of symptoms and conditions during analysis. The analysis unit uses generative AI to apply different analysis algorithms according to the category of symptoms and conditions during analysis. For example, in the case of cardiac symptoms, the analysis unit applies specialized analysis algorithms. In the case of respiratory symptoms, the analysis unit selects appropriate analysis algorithms. Furthermore, in the case of trauma-related conditions, the analysis unit may use specific analysis algorithms to analyze information. By applying different analysis algorithms according to the category of symptoms and conditions, appropriate information can be provided. Specifically, the analysis unit receives as input symptom and condition data from the reception unit (e.g., text “chest pain,” image: chest X-ray, vital: heart rate 120 bpm). The analysis unit first automatically determines the symptom category (e.g., cardiac, respiratory, trauma, neurological, etc.) using large language models or rule-based classifiers. According to the category determination result, the analysis unit automatically selects and applies specialized analysis pipelines, such as ECG waveform analysis algorithms (e.g., RNN or CNN-LSTM hybrid) for cardiac symptoms, respiratory sound spectrum analysis or oxygen saturation time-series analysis for respiratory symptoms, image anomaly detection CNN or fall pattern analysis algorithms for trauma-related conditions. Examples of input include “symptom: chest pain, ECG waveform,” “symptom: dyspnea, respiratory sound data,” “condition: fall, trauma image,” etc. The analysis unit outputs severity scores, emergency labels, and recommended rescue measures using category-specific models. Example outputs include “cardiac category: severity 0.95, emergency: immediate transport,” “respiratory category: severity 0.80, emergency: observation,” “trauma category: severity 0.70, emergency: transport recommended,” etc. These outputs are passed to subsequent provision units or decision units and used to optimize rescue arrangements and notification content. As a technical effect, the analysis unit achieves simultaneous improvement of analysis accuracy, specialization, and speed by combining AI-based automatic category determination and automatic switching of algorithms, which was difficult with conventional uniform application of analysis algorithms, thereby enabling high-precision, symptom-specific analysis. Application fields include emergency medical call apps, remote diagnostic support systems, and mass notification platforms during disasters. Thus, the analysis unit achieves improvement of computer technology by controlling analysis algorithms according to category, beyond mere automation of human tasks.

The analysis unit is capable of estimating the user's emotions and adjusting the display method of analysis results based on the estimated emotions. The analysis unit uses generative AI to estimate the user's emotions and adjust the display method of analysis results based on the estimated emotions. For example, if the user is nervous, the analysis unit provides a simple and highly visible display method. If the user is relaxed, the analysis unit provides a display method including detailed information. Furthermore, if the user is in a hurry, the analysis unit provides a display method that highlights key points. By adjusting the display method of analysis results according to the user's emotions, important information can be provided quickly. Emotion estimation is realized using emotion engines or generative AI with emotion estimation functions. Generative AI may be a text generative AI (e.g., LLM) or a multimodal generative AI, but is not limited thereto. Specifically, the analysis unit receives as input voice data obtained from the user (e.g., 30 seconds of voice waveform), text data (e.g., natural language sentences such as “I'm suffering”), image data (e.g., facial photos), and vital data (e.g., heart rate, skin conductance). The analysis unit performs acoustic feature extraction (e.g., MFCC, pitch) on voice data, expression feature extraction using CNN on image data, and emotion classification (e.g., nervous, relaxed, hurried) using large language models on text data. Vital data is analyzed by time-series analysis models to calculate stress indices. These features are input to a multimodal transformer, which outputs emotion categories (e.g., nervous, relaxed, hurried) and confidence scores. Examples of input include “voice: fast speech,” “text: ‘in a hurry’,” “image: furrowed brow,” “heart rate: 115 bpm,” etc. The analysis unit uses this emotion estimation result to dynamically change the display method of analysis results (e.g., UI layout, amount of information, highlighted items, color coding). For example, in a nervous state, only the most important items are prominently displayed and detailed information is collapsed. In a relaxed state, detailed analysis results, graphs, and past comparison data are also displayed. In a hurry, only key points are displayed immediately in bullet points. Example outputs include “display format: simple, highlighted item: severity,” “display format: detailed, with graphs,” “display format: key points only,” etc. These display controls are passed to user interface generation modules and used for automatic optimization of terminal screens and notification content. As a technical effect, the analysis unit responds immediately to psychological changes in users, which were difficult to handle with conventional uniform display methods, and achieves significant improvement of information transmission efficiency, visibility, and user satisfaction by combining AI-based emotion estimation and display control. Application fields include emergency medical call apps, mass notification systems during disasters, and elderly monitoring terminals. Thus, the analysis unit achieves improvement of computer technology by controlling display methods according to user status, beyond mere automation of human tasks.

The analysis unit is capable of determining the priority of analysis based on the occurrence time of symptoms and conditions during analysis. The analysis unit uses generative AI to determine the priority of analysis based on the occurrence time of symptoms and conditions during analysis. For example, the analysis unit prioritizes analysis of recently occurred symptoms and conditions. The analysis unit may also refer to past symptoms and conditions and prioritize analysis of highly relevant information. Furthermore, the analysis unit may dynamically adjust the priority of analysis according to the occurrence time of symptoms and conditions. By determining the priority of analysis based on the occurrence time of symptoms and conditions, important information can be provided quickly. Specifically, the analysis unit receives as input symptom and condition data from the reception unit (e.g., text “chest pain,” occurrence time “2024-06-01 14:23:10,” time-series vital data). The analysis unit extracts occurrence time and duration as time-series features for each symptom and condition, and uses time-series analysis models (e.g., LSTM, autoregressive models) to score novelty, acuteness, and relevance to past cases. Examples of input include “symptom: chest pain, occurrence time: 2024-06-01 14:23:10,” “condition: fall, occurrence time: 2024-06-01 13:50:00,” etc. The analysis unit places symptoms and conditions with the most recent occurrence at the top of the priority list, and determines the priority of past symptoms and conditions based on relevance scores (e.g., similarity to past chest pain history). Example outputs include “priority 1: chest pain (occurred at 14:23:10),” “priority 2: fall (occurred at 13:50:00),” etc. These priority information are reflected in the processing order of the analysis pipeline and the notification order to subsequent provision units and decision units. As a technical effect, the analysis unit achieves significant improvement of analysis efficiency, speed, and information transmission accuracy by combining AI-based occurrence time analysis and automatic determination of priority, which was difficult with conventional uniform analysis order, thereby enabling time-series-linked priority control. Application fields include emergency medical call apps, mass notification systems during disasters, and corporate health management platforms. Thus, the analysis unit achieves improvement of computer technology by controlling analysis priority according to occurrence time, beyond mere automation of human tasks.

The analysis unit is capable of adjusting the order of analysis based on the relevance of symptoms and conditions during analysis. The analysis unit uses generative AI to adjust the order of analysis based on the relevance of symptoms and conditions during analysis. For example, the analysis unit prioritizes analysis of highly relevant symptoms and conditions. The analysis unit may also analyze the relevance of symptoms and conditions and determine the optimal order of analysis. Furthermore, the analysis unit may dynamically adjust the order of analysis according to the relevance of symptoms and conditions. By adjusting the order of analysis based on the relevance of symptoms and conditions, important information can be provided quickly. Specifically, the analysis unit receives as input multiple symptom and condition data from the reception unit (e.g., chest pain, dyspnea, fall, consciousness disorder). The analysis unit scores the relevance between symptoms and conditions by analyzing co-occurrence patterns (e.g., chest pain plus dyspnea indicates high risk of myocardial infarction), time-series continuity, and similarity to past case databases. Examples of input include “symptoms: chest pain, dyspnea,” “conditions: fall, consciousness disorder,” etc. The analysis unit groups combinations of symptoms and conditions with high relevance and automatically generates an analysis order list. Example outputs include “analysis order 1: chest pain+dyspnea (relevance 0.92),” “analysis order 2: fall+consciousness disorder (relevance 0.85),” etc. These order information are reflected in the processing order of the analysis pipeline and the notification order to subsequent provision units and decision units. As a technical effect, the analysis unit achieves significant improvement of analysis efficiency, speed, and reduction of false reports by combining AI-based relevance analysis and automatic determination of order, which was difficult with conventional uniform analysis order, thereby enabling relevance-linked order control. Application fields include emergency medical call apps, mass notification systems during disasters, and corporate health management platforms. Thus, the analysis unit achieves improvement of computer technology by controlling analysis order according to relevance, beyond mere automation of human tasks.

The provision unit is capable of estimating the user's emotions and adjusting the method of information provision based on the estimated emotions. The provision unit uses generative AI to estimate the user's emotions and adjusts the method of information provision according to the estimated emotions. For example, if the user is nervous, the provision unit provides a simple and highly visible information provision method. If the user is relaxed, the provision unit can provide a method that includes detailed information. Furthermore, if the user is in a hurry, the provision unit can provide a method that focuses on key points. By adjusting the method of information provision according to the user's emotions, important information can be provided quickly. Emotion estimation is realized using an emotion estimation function, for example, by employing an emotion engine or generative AI. Generative AI may include text generation AI (e.g., LLM) or multimodal generative AI, but is not limited thereto. Specifically, the provision unit receives as input voice data obtained from the user (e.g., 30 seconds of audio waveform sampled at 16 kHz), text data (e.g., natural language sentences such as “I feel pain”), image data (e.g., facial photos or expression images), and vital data (e.g., heart rate, skin conductance response). The provision unit performs spectrogram conversion and acoustic feature extraction (e.g., MFCC, pitch, energy) on the voice data, and extracts facial features from the image data using a convolutional neural network (CNN). The text data is processed by a large language model to estimate emotion labels (e.g., nervous, anxious, calm) and emotion scores (e.g., “nervous” with a score of 0.88). The vital data is analyzed by a time-series analysis model (e.g., LSTM) to calculate stress indices and abnormal values. These diverse features are input into a multimodal transformer-based integrated model, which outputs the final emotion estimation result (e.g., emotion category “nervous”, confidence 0.88). Examples of input include “voice: trembling”, “text: ‘I feel pain’”, “image: tense face”, “heart rate: 110 bpm”. Based on this emotion estimation result, the provision unit dynamically changes the information provision method (e.g., UI layout, amount of information, highlighted items, color coding). For example, in a nervous state, only the most important items are prominently displayed, and detailed information is collapsed. In a relaxed state, detailed analysis results, graphs, and historical comparison data are also displayed. In a hurry, only the key points are immediately displayed in a bulleted format. Examples of output include “display format: simple, highlighted item: severity”, “display format: detailed, with graph”, “display format: key points only”. These display controls are passed to the user interface generation module and used for automatic optimization of the terminal screen and notification content. As a technical effect, the provision unit can promptly respond to changes in user psychology, which was difficult with conventional uniform display methods, and greatly improves information transmission efficiency, visibility, and user satisfaction by combining AI-based emotion estimation and display control. Application fields include emergency medical reporting apps, mass notification systems during disasters, and elderly monitoring terminals. Thus, the provision unit achieves an improvement in computer technology as a user state-linked display control, going beyond mere automation of human tasks.

The provision unit is capable of adjusting the content of information provision based on the importance of the emergency call receiving agency or medical institution at the time of information provision. The provision unit uses generative AI to adjust the content of information provision according to the importance of the emergency call receiving agency or medical institution. For example, the provision unit provides detailed information to important emergency call receiving agencies or medical institutions. The provision unit can also provide simplified information to less critical emergency call receiving agencies or medical institutions. Furthermore, the provision unit can dynamically adjust the content of provision according to the importance of the emergency call receiving agency or medical institution. By adjusting the content of provision according to the importance, important information can be provided quickly. Specifically, the provision unit receives as input the importance score set for each emergency call receiving agency or medical institution (e.g., a continuous value from 0 to 1, with 0.9 being the most important) and priority labels (e.g., highest priority, normal, low priority). The provision unit automatically adjusts the amount and detail of information according to the importance score, using structured data such as severity score, urgency label, vital data, medical history, and location information received from the analysis unit. For example, when the importance is high, all items (e.g., vital time series, image features, past history, recommended actions) are sent in detail, and when the importance is low, only the key points (e.g., severity, urgency, location information) are sent in a simplified manner. Examples of output include “content: detailed (all items sent)” and “content: simplified (key points only)”. These outputs are transmitted in real time using communication protocols (e.g., HTTPS, MQTT), and format conversion and priority assignment are performed according to the requirements of the receiving system. As a technical effect, the provision unit realizes resource optimization and prevention of information overload or insufficiency, which were difficult with conventional uniform information provision, by combining AI-based importance determination and automatic adjustment of content, thereby greatly improving communication efficiency, information transmission accuracy, and on-site responsiveness. Application fields include urban emergency medical care, mass notification during disasters, and remote medical support. Thus, the provision unit achieves an improvement in computer technology as importance-linked information provision optimization, going beyond mere automation of human tasks.

The provision unit is capable of applying different provision algorithms according to the category of the emergency call receiving agency or medical institution at the time of information provision. The provision unit uses generative AI to apply different provision algorithms according to the category of the emergency call receiving agency or medical institution. For example, the provision unit applies specialized provision algorithms to emergency medical institutions. The provision unit can also select appropriate provision algorithms for general medical institutions. Furthermore, the provision unit can dynamically adjust the provision algorithm according to the category of the emergency call receiving agency or medical institution. By doing so, appropriate information can be provided according to the category. Specifically, the provision unit receives as input the category information of the receiving institution (e.g., emergency medical, general practice, specialized practice, disaster response headquarters). The provision unit processes and extracts symptom and condition data received from the analysis unit using provision algorithms optimized for each category (e.g., for emergency medical: severity, urgency, detailed vital data; for general practice: focus on medical history and medication information; for disaster response: location information, group status, scale of damage). For example, detailed data such as “severity 0.95, urgency: immediate transport, heart rate data [120, 130, 125]” are sent to emergency medical institutions, while basic information such as “medical history: hypertension, medication: antihypertensive” is sent to general medical institutions. Examples of output include “provision algorithm: for emergency medical” and “provision algorithm: for general practice”. These algorithm selections are linked with AI-based automatic category determination, realizing optimization of provision content, enhancement of specialization, and efficiency of information transmission. As a technical effect, the provision unit realizes category-specific information optimization, which was difficult with conventional uniform information provision, by combining AI-based category determination and automatic switching of algorithms, thereby greatly improving on-site responsiveness, specialization, and communication efficiency. Application fields include emergency medical reporting, remote diagnostic support, and mass notification during disasters. Thus, the provision unit achieves an improvement in computer technology as category-linked information provision algorithm control, going beyond mere automation of human tasks.

The provision unit is capable of estimating the user's emotions and determining the priority of information provision based on the estimated emotions. The provision unit uses generative AI to estimate the user's emotions and determines the priority of information provision according to the estimated emotions. For example, if the user is nervous, the provision unit prioritizes the provision of important information. If the user is relaxed, the provision unit can provide detailed information. Furthermore, if the user is in a hurry, the provision unit can quickly provide the most important information. By determining the priority of information provision according to the user's emotions, important information can be provided quickly. Emotion estimation is realized using an emotion estimation function, for example, by employing an emotion engine or generative AI. Generative AI may include text generation AI (e.g., LLM) or multimodal generative AI, but is not limited thereto. Specifically, the provision unit receives as input voice data, text data, image data, and vital data obtained from the user. The provision unit performs acoustic feature extraction on the voice data, facial feature extraction using CNN on the image data, emotion classification using a large language model on the text data, and stress index calculation using a time-series analysis model on the vital data. These features are input into a multimodal transformer, which outputs emotion categories (e.g., nervous, relaxed, hurried) and confidence scores. Examples of output include “emotion: nervous, confidence 0.88” and “emotion: relaxed, confidence 0.72”. Based on this emotion estimation result, the provision unit automatically determines the priority of information provision (e.g., most important items→key points→detailed information) and dynamically controls the notification order and screen display order for emergency call receiving agencies or medical institutions. As a technical effect, the provision unit can promptly respond to changes in user psychology, which was difficult with conventional uniform information provision order, and greatly improves information transmission efficiency, speed, and on-site responsiveness by combining AI-based emotion estimation and priority control. Application fields include emergency medical reporting apps, mass notification systems during disasters, and elderly monitoring terminals. Thus, the provision unit achieves an improvement in computer technology as user state-linked information provision priority control, going beyond mere automation of human tasks.

The provision unit is capable of adjusting the content of information provision based on the geographic location information of the emergency call receiving agency or medical institution at the time of information provision. The provision unit uses generative AI to adjust the content of information provision by considering the geographic location information of the emergency call receiving agency or medical institution. For example, the provision unit provides optimal information based on the geographic location information of the emergency call receiving agency or medical institution. The provision unit can also provide detailed information to geographically close emergency call receiving agencies or medical institutions. Furthermore, the provision unit can refer to the geographic location information and dynamically adjust the content of provision. By considering the geographic location information, optimal information can be provided. Specifically, the provision unit receives as input the location information of the emergency call receiving agency or medical institution (e.g., latitude/longitude, address, area code). The provision unit collaborates with external data such as the user's current location, planned transport destination, distance calculation, region-specific medical resource status (e.g., bed availability, presence of specialists), and disaster area information, and automatically generates information provision content optimized for geographic conditions (e.g., detailed vital and location information for the nearest institution, key points only for remote locations). Examples of output include “content: detailed (for nearest institution)” and “content: simplified (for remote location)”. These outputs are transmitted in real time using communication protocols, and the amount and priority of information are automatically adjusted according to the geographic conditions of the receiving side. As a technical effect, the provision unit realizes geographic optimization, which was difficult with conventional uniform information provision, by combining AI-based location information analysis and automatic adjustment of content, thereby greatly improving transport efficiency, on-site responsiveness, and information transmission accuracy. Application fields include urban and rural emergency medical care, wide-area transport during disasters, and remote medical support. Thus, the provision unit achieves an improvement in computer technology as geographic information-linked information provision optimization, going beyond mere automation of human tasks.

The provision unit is capable of adjusting the content of information provision by referring to related literature of the emergency call receiving agency or medical institution at the time of information provision. The provision unit uses generative AI to adjust the content of information provision by referring to related literature of the emergency call receiving agency or medical institution. For example, the provision unit provides optimal information based on related literature of the emergency call receiving agency or medical institution. The provision unit can also provide detailed information to emergency call receiving agencies or medical institutions by referring to related literature. Furthermore, the provision unit can dynamically adjust the content of provision by referring to related literature. By referring to related literature, optimal information can be provided. Specifically, the provision unit refers to literature databases associated with each emergency call receiving agency or medical institution, such as medical guidelines, treatment protocols, and the latest research papers. The provision unit matches symptom and condition data received from the analysis unit with the content of related literature (e.g., emergency response procedures, disease-specific priority information, transport criteria), and automatically extracts and adds recommended information and precautions based on the literature. For example, in the case of suspected myocardial infarction, rules such as “send ECG data and detailed vital information as mandatory items based on the 2024 guideline” are applied. Examples of output include “content: guideline-compliant” and “content: latest paper referenced”. These outputs automatically add literature reference history and recommendation basis at the time of information provision, and are also used for decision support and record management at the receiving medical institution. As a technical effect, the provision unit realizes evidence-based information optimization, which was difficult with conventional experience-based or subjective judgment, by combining AI-based literature analysis and automatic adjustment of content, thereby greatly improving standardization, accuracy, and record efficiency in medical settings. Application fields include emergency medical reporting, remote diagnostic support, and mass notification during disasters. Thus, the provision unit achieves an improvement in computer technology as literature-linked information provision optimization, going beyond mere automation of human tasks.

The decision unit is capable of estimating the user's emotions and adjusting the method of determining deployment of emergency vehicles or transport routes based on the estimated emotions. The decision unit uses generative AI to estimate the user's emotions and adjusts the method of determining deployment of emergency vehicles or transport routes according to the estimated emotions. For example, if the user is nervous, the decision unit prioritizes rapid deployment and the shortest route. If the user is relaxed, the decision unit can provide detailed route information. Furthermore, if the user is in a hurry, the decision unit can select the most efficient deployment and route. By adjusting the method of determining deployment and transport routes according to the user's emotions, rapid response becomes possible. Emotion estimation is realized using an emotion estimation function, for example, by employing an emotion engine or generative AI. Generative AI may include text generation AI (e.g., LLM) or multimodal generative AI, but is not limited thereto. Specifically, the decision unit receives as input voice data obtained from the user (e.g., 30 seconds of audio waveform sampled at 16 kHz), text data (e.g., natural language sentences such as “Help, I feel pain”), image data (e.g., facial photos or expression images), and vital data (e.g., heart rate, skin conductance response). The decision unit performs spectrogram conversion and acoustic feature extraction (e.g., MFCC, pitch, energy) on the voice data, and extracts facial features from the image data using a convolutional neural network. The text data is processed by a large language model to estimate emotion labels (e.g., nervous, anxious, calm) and emotion scores (e.g., “nervous” with a score of 0.88). The vital data is analyzed by a time-series analysis model (e.g., LSTM) to calculate stress indices and abnormal values. These diverse features are input into a multimodal transformer-based integrated model, which outputs the final emotion estimation result (e.g., emotion category “nervous”, confidence 0.88). Examples of input include “voice: trembling”, “text: ‘I feel pain’”, “image: tense face”, “heart rate: 110 bpm”. Based on this emotion estimation result, the decision unit dynamically adjusts the parameters of the deployment and transport route determination algorithm. For example, in a nervous state, immediacy of deployment instructions is prioritized, and the cost function of the shortest path search algorithm (e.g., A* algorithm) is weighted heavily for “arrival time priority”. In a relaxed state, detailed route information (e.g., waypoints, estimated arrival time, traffic condition explanation) is added, and information provision to the user and medical institution is enhanced. In a hurry, the algorithm branches to prioritize efficiency (e.g., simultaneous deployment of multiple vehicles, immediate selection of congestion avoidance routes). Examples of output include “deployment instruction: vehicle ID123, dispatch time 14:25, route A (nervous state, shortest)” and “deployment instruction: vehicle ID456, dispatch time 14:30, route B (relaxed state, with detailed explanation)”. These outputs are automatically sent to the emergency vehicle operation system and medical institutions, and are used for rapid decision-making and transport preparation on site. As a technical effect, the decision unit can promptly respond to changes in user psychology and on-site conditions, which was difficult with conventional uniform deployment and transport route determination, and greatly improves the speed, flexibility, and on-site responsiveness of deployment and transport by combining AI-based emotion estimation and automatic optimization of decision algorithms. Application fields include urban emergency medical care, wide-area transport during disasters, remote medical support, and corporate safety management systems. Thus, the decision unit achieves an improvement in computer technology as user state-linked deployment and transport decision-making, going beyond mere automation of human tasks.

The decision unit is capable of selecting an optimal determination method by referring to past deployment history when determining deployment of emergency vehicles or transport routes. The decision unit uses generative AI to select an optimal determination method by referring to past deployment history when determining deployment of emergency vehicles or transport routes. For example, the decision unit selects the optimal deployment method based on past deployment history. The decision unit can also select the optimal route by referring to past transport route history. Furthermore, the decision unit can analyze past deployment history and select the most efficient deployment method. By referring to past deployment history, the optimal deployment method can be selected. Specifically, the decision unit obtains as input a database of past emergency vehicle deployment and transport route history (e.g., structured data including deployment date and time, vehicle ID, dispatch location, transport destination, required time, success rate, congestion occurrence, patient status, transport result). The decision unit preprocesses these history data as time-series vectors and categorical features, and applies algorithms such as frequency analysis (e.g., deployment patterns in specific areas), clustering (e.g., transport tendencies by symptom or condition), and performance evaluation (e.g., average transport time, success rate). Generative AI (e.g., large language models or time-series analysis models) automatically extracts past cases similar to the current situation based on these features, and outputs optimal deployment methods (e.g., route with shortest transport time, deployment pattern with proven congestion avoidance) and recommended routes. Examples of input include “2024-06-01 14:23:10, vehicle ID123, dispatch location A, transport destination B, required time 15 minutes, success” and “2024-05-20 08:10:00, vehicle ID456, dispatch location C, transport destination D, required time 25 minutes, congestion occurred”. Examples of output include “recommended deployment method: vehicle ID123, route A→B (shortest in history)” and “recommended route: C→D (congestion avoided)”. These outputs are reflected in the deployment and transport algorithms of the decision unit, automating optimal decision-making according to on-site conditions. Furthermore, the decision unit can automatically detect past failure cases and delay patterns, and propose similar risk avoidance measures (e.g., simultaneous deployment of backup vehicles, pre-selection of detour routes). As a technical effect, the decision unit realizes evidence-based optimization, which was difficult with conventional experience-based or subjective judgment, by combining AI-based history analysis and automated decision-making, thereby greatly improving transport efficiency, success rate, and on-site responsiveness. Application fields include urban emergency medical care, wide-area transport during disasters, remote medical support, and corporate safety management systems. Thus, the decision unit achieves an improvement in computer technology as history-linked deployment and transport optimization, going beyond mere automation of human tasks.

The decision unit is capable of adjusting the determination content based on current traffic conditions when determining deployment of emergency vehicles or transport routes. The decision unit uses generative AI to adjust the determination content according to current traffic conditions when determining deployment of emergency vehicles or transport routes. For example, the decision unit selects the optimal transport route based on current traffic congestion information. The decision unit can also select a rapid deployment method by considering current traffic conditions. Furthermore, the decision unit can refer to current traffic information and determine the optimal deployment and transport route. By determining the optimal deployment and transport route based on current traffic conditions, rapid response becomes possible. Specifically, the decision unit receives as input real-time traffic condition data obtained from traffic information APIs or geographic information systems (e.g., congestion level for each road, accident locations, construction zones, estimated travel time, traffic regulation information). The decision unit preprocesses these traffic data as graph structures (e.g., road network nodes and edges, each edge assigned travel cost or congestion score), and applies shortest path search algorithms (e.g., Dijkstra's algorithm, A* algorithm) and real-time route recalculation algorithms. Generative AI (e.g., large language models or reinforcement learning models) considers traffic pattern changes and past transport records, and outputs optimal deployment and transport routes (e.g., congestion avoidance routes, detour routes around accident sites, simultaneous deployment patterns for multiple vehicles). Examples of input include “Point A to Hospital B: normally 15 minutes, currently congested +10 minutes” and “Road C: accident, closed”. Examples of output include “recommended route: A→D→B (congestion avoided, required time 18 minutes)” and “deployment instruction: vehicle ID789, dispatch time 14:28, using detour route”. These outputs are reflected in the deployment and transport algorithms of the decision unit, automating optimal decision-making according to on-site conditions. Furthermore, the decision unit can monitor changes in traffic conditions in real time during transport and immediately recalculate routes or change deployment as needed. As a technical effect, the decision unit realizes real-time traffic-linked optimization, which was difficult with conventional static route selection or manual judgment by human operators, by combining AI-based traffic data analysis and automated decision-making, thereby greatly improving transport efficiency, speed, and on-site responsiveness. Application fields include urban emergency medical care, wide-area transport during disasters, remote medical support, and corporate safety management systems. Thus, the decision unit achieves an improvement in computer technology as traffic information-linked deployment and transport optimization, going beyond mere automation of human tasks.

The decision unit is capable of estimating the user's emotions and determining the priority of deployment of emergency vehicles or transport routes based on the estimated emotions. The decision unit uses generative AI to estimate the user's emotions and determines the priority of deployment of emergency vehicles or transport routes according to the estimated emotions. For example, if the user is nervous, the decision unit prioritizes rapid deployment and the shortest route. If the user is relaxed, the decision unit can provide detailed route information. Furthermore, if the user is in a hurry, the decision unit can select the most efficient deployment and route. By determining the priority of deployment and transport routes according to the user's emotions, rapid response becomes possible. Emotion estimation is realized using an emotion estimation function, for example, by employing an emotion engine or generative AI. Generative AI may include text generation AI (e.g., LLM) or multimodal generative AI, but is not limited thereto. Specifically, the decision unit receives as input voice data (e.g., 30 seconds of audio waveform), text data (e.g., natural language sentences such as “Help, I feel pain”), image data (e.g., facial photos or expression images), and vital data (e.g., heart rate, skin conductance response) obtained from the user. The decision unit performs acoustic feature extraction (e.g., MFCC, pitch) on the voice data, facial feature extraction using a convolutional neural network on the image data, emotion classification (e.g., nervous, relaxed, hurried) using a large language model on the text data, and stress index calculation using a time-series analysis model on the vital data. These features are input into a multimodal transformer, which outputs emotion categories (e.g., nervous, relaxed, hurried) and confidence scores. Examples of output include “emotion: nervous, confidence 0.88” and “emotion: relaxed, confidence 0.72”. Based on this emotion estimation result, the decision unit automatically determines the priority of deployment and transport routes (e.g., shortest route→detailed route→efficiency-focused route), and dynamically controls the notification order and screen display order for emergency vehicle operation systems and medical institutions. For example, in a nervous state, “shortest route and immediate deployment” is prioritized; in a relaxed state, “route with detailed explanation” is prioritized; and in a hurry, “efficiency-focused route” is immediately selected. These priority information are used for rapid decision-making and transport preparation on site. As a technical effect, the decision unit can promptly respond to changes in user psychology, which was difficult with conventional uniform deployment and transport order, and greatly improves transport efficiency, speed, and on-site responsiveness by combining AI-based emotion estimation and priority control. Application fields include urban emergency medical care, wide-area transport during disasters, remote medical support, and corporate safety management systems. Thus, the decision unit achieves an improvement in computer technology as user state-linked deployment and transport priority control, going beyond mere automation of human tasks.

The decision unit is capable of selecting an appropriate determination method based on geographic location information when determining deployment of emergency vehicles or transport routes. The decision unit uses generative AI to select the optimal determination method by considering geographic location information when determining deployment of emergency vehicles or transport routes. For example, the decision unit prioritizes deployment of emergency vehicles that are geographically close. The decision unit can also select the optimal transport route based on geographic location information. Furthermore, the decision unit can refer to geographic location information and determine the optimal deployment and transport route. By considering geographic location information, the optimal deployment and transport route can be determined. Specifically, the decision unit receives as input latitude and longitude data obtained from GPS sensors of user terminals and emergency vehicles, geographic information systems (e.g., user location 35.6895, 139.6917; vehicle location 35.6900, 139.7000), area codes, and location information of the destination medical institution. The decision unit preprocesses these geographic information as spatial vectors and graph structures, and applies spatial analysis algorithms such as distance calculation between user, vehicle, and medical institution, estimated arrival time, and consideration of geographic obstacles (e.g., rivers, mountains, closed roads). Generative AI (e.g., large language models or geographic information linkage modules) combines geographic conditions and past transport records to output optimal deployment methods (e.g., immediate deployment of the nearest vehicle, simultaneous deployment of multiple vehicles) and transport routes (e.g., shortest distance route, safe route during disasters). Examples of input include “user location: 35.6895, 139.6917; vehicle location: 35.6900, 139.7000” and “destination: Hospital A (35.6880, 139.6950)”. Examples of output include “recommended deployment: vehicle ID123 (distance 0.8 km, estimated arrival 2 minutes)” and “recommended route: user→Hospital A (shortest distance)”. These outputs are reflected in the deployment and transport algorithms of the decision unit, automating optimal decision-making according to on-site conditions. Furthermore, the decision unit can automatically detect geographic obstacles and disaster areas, and propose risk avoidance routes or alternative transport destinations. As a technical effect, the decision unit realizes geographic optimization, which was difficult with conventional uniform deployment and transport determination, by combining AI-based location information analysis and automated decision-making, thereby greatly improving transport efficiency, on-site responsiveness, and safety. Application fields include urban and rural emergency medical care, wide-area transport during disasters, remote medical support, and corporate safety management systems. Thus, the decision unit achieves an improvement in computer technology as geographic information-linked deployment and transport optimization, going beyond mere automation of human tasks.

The decision unit is capable of adjusting the determination content by referring to related literature when determining deployment of emergency vehicles or transport routes. The decision unit uses generative AI to adjust the determination content by referring to related literature when determining deployment of emergency vehicles or transport routes. For example, the decision unit selects the optimal deployment method based on related literature. The decision unit can also select the optimal transport route by referring to related literature. Furthermore, the decision unit can refer to related literature and determine the optimal deployment and transport route. By referring to related literature, the optimal deployment and transport route can be determined. Specifically, the decision unit refers to literature databases related to emergency vehicle deployment and transport, such as medical guidelines, disaster transport protocols, the latest research papers, and administrative notifications. The decision unit matches symptom and condition data received from the analysis unit or provision unit with the content of related literature (e.g., disease-specific transport criteria, priority deployment rules, recommended special routes during disasters), and automatically extracts and adds recommended deployment methods, transport routes, and precautions based on the literature. Generative AI (e.g., large language models or literature search modules) calculates matching scores between symptoms/conditions and literature content, supporting evidence-based decision-making. Examples of input include “symptom: suspected myocardial infarction, refer to 2024 guideline” and “situation: disaster transport, disaster response headquarters notification”. Examples of output include “recommended deployment: guideline-compliant (vehicle ID123, route A)” and “recommended route: latest paper referenced (safety prioritized)”. These outputs are reflected in the deployment and transport algorithms of the decision unit, and are also used for standardization, record management, and presentation of decision rationale on site. As a technical effect, the decision unit realizes evidence-based optimization, which was difficult with conventional experience-based or subjective judgment, by combining AI-based literature analysis and automated decision-making, thereby greatly improving transport accuracy, standardization, and on-site responsiveness. Application fields include emergency medical reporting, wide-area transport during disasters, remote medical support, and corporate safety management systems. Thus, the decision unit achieves an improvement in computer technology as literature-linked deployment and transport optimization, going beyond mere automation of human tasks.

The monitoring unit is capable of estimating the user's emotions and adjusting the frequency of monitoring based on the estimated emotions. The monitoring unit uses generative AI to estimate the user's emotions and adjusts the frequency of monitoring according to the estimated emotions. For example, if the user is nervous, the monitoring unit increases the frequency of monitoring. If the user is relaxed, the monitoring unit can decrease the frequency of monitoring. Furthermore, if the user is in a hurry, the monitoring unit can perform rapid monitoring. By adjusting the frequency of monitoring according to the user's emotions, important information can be quickly grasped. Emotion estimation is realized using an emotion estimation function, for example, by employing an emotion engine or generative AI. Generative AI may include text generation AI (e.g., LLM) or multimodal generative AI, but is not limited thereto. Specifically, the monitoring unit receives as input voice data obtained from the user terminal (e.g., 30 seconds of audio waveform sampled at 16 kHz), text data (e.g., natural language sentences such as “I am anxious”, “I am calm”), image data (e.g., facial photos or expression images), and vital data (e.g., heart rate, skin conductance response). The monitoring unit performs spectrogram conversion and acoustic feature extraction (e.g., MFCC, pitch, energy) on the voice data, and extracts facial features from the image data using a convolutional neural network. The text data is processed by a large language model to estimate emotion labels (e.g., nervous, anxious, calm) and emotion scores (e.g., “nervous” with a score of 0.88). The vital data is analyzed by a time-series analysis model (e.g., LSTM) to calculate stress indices and abnormal values. These diverse features are input into a multimodal transformer-based integrated model, which outputs the final emotion estimation result (e.g., emotion category “nervous”, confidence 0.88). Examples of input include “voice: trembling”, “text: ‘I am anxious’”, “image: tense face”, “heart rate: 110 bpm”. Based on this emotion estimation result, the monitoring unit dynamically adjusts the parameters of the monitoring frequency control module. For example, in a nervous state, the monitoring interval is shortened to every 1 minute; in a relaxed state, it is extended to every 10 minutes. In a hurry, the mode is switched to high-speed mode, which immediately acquires only important vital items. Examples of output include “monitoring frequency: 1-minute interval (nervous state)” and “monitoring frequency: 10-minute interval (relaxed state)”. These frequency controls are reflected in the sensor data acquisition module and abnormality detection module, and the monitoring strategy is optimized in real time. As a technical effect, the monitoring unit can promptly respond to changes in user psychology and urgency, which was difficult with conventional uniform monitoring frequency settings, and simultaneously realizes monitoring efficiency, abnormality detection accuracy, and resource consumption optimization by combining AI-based emotion estimation and frequency control. Application fields include emergency medical reporting apps, elderly monitoring terminals, remote health management for chronic disease patients, and mass health monitoring during disasters. Thus, the monitoring unit achieves an improvement in computer technology as user state-linked monitoring frequency control, going beyond mere automation of human tasks.

The monitoring unit is capable of selecting an optimal monitoring method by referring to the user's past health data during monitoring. The monitoring unit uses generative AI to select an optimal monitoring method by referring to the user's past health data during monitoring. For example, the monitoring unit selects the optimal monitoring method based on the user's past health data. The monitoring unit can also prioritize monitoring of important information by referring to the user's past health data. Furthermore, the monitoring unit can analyze the user's health data and select the optimal monitoring method. By referring to the user's past health data, the optimal monitoring method can be selected. Specifically, the monitoring unit obtains as input a health database accumulated for each user (e.g., time-series data for the past year such as heart rate, blood pressure, blood glucose, body temperature, medical history, medication history, past abnormality detection history). The monitoring unit preprocesses these data as time-series vectors and categorical features, and applies algorithms such as frequency analysis (e.g., frequent heart rate abnormalities in the past month), clustering (e.g., abnormal tendencies by symptom), and pattern extraction (e.g., blood pressure rises at specific times). Generative AI (e.g., large language models or time-series analysis models) recommends the optimal monitoring method for each user (e.g., focused monitoring of heart rate, real-time monitoring of blood glucose, monitoring of body temperature only at night), monitoring frequency, and priority of monitoring items based on these features. Examples of input include “2024-06-01 14:23:10, heart rate 120 bpm, blood pressure 150/95 mmHg, medical history: hypertension” and “2024-05-20 08:10:00, blood glucose 180 mg/dL, medication: insulin”. Examples of output include “recommended monitoring method: heart rate every 1 minute, blood pressure every 10 minutes” and “priority monitoring items: blood glucose, heart rate”. These outputs are reflected in the sensor control module and abnormality detection module of the monitoring unit, and a user-optimized monitoring strategy is automatically executed. Furthermore, based on past abnormal occurrence patterns, preventive monitoring enhancement and automatic adjustment of alert thresholds are also possible. As a technical effect, the monitoring unit realizes user-specific optimization, which was difficult with conventional uniform monitoring methods, by combining AI-based history analysis and automatic monitoring optimization, thereby greatly improving monitoring efficiency, abnormality detection accuracy, and user satisfaction. Application fields include remote health management for chronic disease patients, employee health monitoring in companies, home medical support, and health monitoring during disasters. Thus, the monitoring unit achieves an improvement in computer technology as history-linked monitoring optimization, going beyond mere automation of human tasks.

The monitoring unit is capable of adjusting the content of monitoring based on the user's current living conditions during monitoring. The monitoring unit uses generative AI to adjust the content of monitoring according to the user's current living conditions during monitoring. For example, the monitoring unit adjusts the content of monitoring by considering the user's current living conditions. The monitoring unit can also prioritize monitoring of important information based on the user's living conditions. Furthermore, the monitoring unit can refer to the user's living conditions and propose optimal monitoring content. By adjusting the content of monitoring according to the user's current living conditions, important information can be quickly grasped. Specifically, the monitoring unit receives as input living condition data obtained from user terminals or wearable devices (e.g., step count and exercise intensity from activity trackers, sleep time and quality from sleep sensors, meal content from diet recording apps, outing schedule from calendar integration). The monitoring unit preprocesses these data as time-series vectors and categorical features, and applies algorithms such as lifestyle pattern analysis (e.g., high activity during the day, sleep at night), abnormal behavior detection (e.g., sudden decrease in activity, sleep deprivation), and lifestyle event detection (e.g., travel, long outings). Generative AI (e.g., large language models or time-series analysis models) recommends monitoring content optimized for the user's current living conditions (e.g., focused monitoring of heart rate and fall detection during exercise, monitoring of respiration and heart rate variability during sleep, enhanced monitoring of location information and fall detection during outings) and priority of monitoring items based on these features. Examples of input include “activity: 10,000 steps, sleep time: 6 hours, outing schedule: 14:00-18:00” and “meal content: high salt, exercise intensity: high”. Examples of output include “priority monitoring items: heart rate, fall detection (during exercise)” and “monitoring content: respiration, heart rate variability (during sleep)”. These outputs are reflected in the sensor control module and abnormality detection module of the monitoring unit, and a dynamic monitoring strategy according to living conditions is automatically executed. As a technical effect, the monitoring unit can promptly respond to changes in living conditions, which was difficult with conventional uniform monitoring content settings, and greatly improves monitoring efficiency, abnormality detection accuracy, and user satisfaction by combining AI-based living data analysis and automatic optimization of monitoring content. Application fields include elderly monitoring, home medical care, health management for chronic disease patients, and corporate health monitoring. Thus, the monitoring unit achieves an improvement in computer technology as living condition-linked monitoring content optimization, going beyond mere automation of human tasks.

The monitoring unit is capable of estimating the user's emotions and determining the priority of monitoring based on the estimated emotions. The monitoring unit uses generative AI to estimate the user's emotions and determines the priority of monitoring according to the estimated emotions. For example, if the user is nervous, the monitoring unit prioritizes monitoring of important information. If the user is relaxed, the monitoring unit can monitor detailed information. Furthermore, if the user is in a hurry, the monitoring unit can quickly monitor the most important information. By determining the priority of monitoring according to the user's emotions, important information can be quickly grasped. Emotion estimation is realized using an emotion estimation function, for example, by employing an emotion engine or generative AI. Generative AI may include text generation AI (e.g., LLM) or multimodal generative AI, but is not limited thereto. Specifically, the monitoring unit receives as input voice data, text data, image data, and vital data obtained from the user terminal. The monitoring unit performs acoustic feature extraction (e.g., MFCC, pitch) on the voice data, facial feature extraction using a convolutional neural network on the image data, emotion classification (e.g., nervous, relaxed, hurried) using a large language model on the text data, and stress index calculation using a time-series analysis model on the vital data. These features are input into a multimodal transformer, which outputs emotion categories (e.g., nervous, relaxed, hurried) and confidence scores. Examples of output include “emotion: nervous, confidence 0.88” and “emotion: relaxed, confidence 0.72”. Based on this emotion estimation result, the monitoring unit automatically determines the priority of monitoring items in the monitoring priority determination module. For example, in a nervous state, the most important items such as “heart rate”, “consciousness level”, and “respiratory rate” are monitored first; in a relaxed state, detailed items such as “medical history” and “medication information” are also monitored sequentially; and in a hurry, only the minimum required items are monitored immediately. These priority information are passed to the sensor control module and abnormality detection module, and are used for dynamic optimization of the monitoring screen and notification content. As a technical effect, the monitoring unit can promptly respond to changes in user psychology during emergencies, which was difficult with conventional uniform monitoring order, and greatly improves monitoring efficiency, abnormality detection accuracy, and information transmission speed by combining AI-based emotion estimation and priority control. Application fields include emergency medical reporting apps, mass health monitoring during disasters, elderly monitoring terminals, and corporate health management systems. Thus, the monitoring unit achieves an improvement in computer technology as user state-linked monitoring priority control, going beyond mere automation of human tasks.

The monitoring unit is capable of selecting an optimal monitoring method by considering the user's geographic location information during monitoring. The monitoring unit uses generative AI to select an optimal monitoring method by considering the user's geographic location information during monitoring. For example, the monitoring unit selects the optimal monitoring method based on the user's geographic location information. The monitoring unit can also prioritize monitoring of geographically relevant information. Furthermore, the monitoring unit can refer to the user's geographic location information and propose optimal monitoring methods. By considering the user's geographic location information, the optimal monitoring method can be selected. Specifically, the monitoring unit receives as input latitude and longitude data obtained from the GPS sensor of the user terminal or network location information API (e.g., 35.6895, 139.6917). The monitoring unit collaborates with a geographic information system (GIS) database to obtain external data such as a list of nearby medical institutions, region-specific infectious disease outbreak information, and disaster occurrence status. Generative AI (e.g., large language models or geographic information linkage modules) combines location information and external data to recommend monitoring methods (e.g., focused monitoring of body temperature and respiratory rate in infectious disease outbreak areas, enhanced detection of falls and injuries in disaster areas, monitoring of location information and battery level in remote areas) and priority of monitoring items. Examples of input include “location: 35.6895, 139.6917, disaster: earthquake occurred” and “location: 34.6937, 135.5023, infectious disease outbreak: influenza”. Examples of output include “recommended monitoring method: focused on body temperature and respiratory rate (infectious disease outbreak area)” and “priority monitoring items: fall detection, injury (disaster area)”. These outputs are reflected in the sensor control module and abnormality detection module of the monitoring unit, and a dynamic monitoring strategy according to geographic conditions is automatically executed. As a technical effect, the monitoring unit can promptly respond to regional characteristics, which was difficult with conventional uniform monitoring method settings, and greatly improves monitoring efficiency, abnormality detection accuracy, and regional medical collaboration by combining AI-based location information analysis and automatic optimization of monitoring methods. Application fields include emergency reporting apps, mass health monitoring during disasters, health management apps for travelers, and remote health monitoring. Thus, the monitoring unit achieves an improvement in computer technology as geographic information-linked monitoring optimization, going beyond mere automation of human tasks.

The monitoring unit is capable of analyzing the user's social media activity and adjusting the content of monitoring during monitoring. The monitoring unit uses generative AI to analyze the user's social media activity and adjust the content of monitoring during monitoring. For example, the monitoring unit extracts information related to the current situation or symptoms from the user's social media posts and adjusts the content of monitoring. The monitoring unit can also analyze the user's social media activity and prioritize monitoring of information related to emergencies. Furthermore, the monitoring unit can refer to the user's social media data and propose optimal monitoring content. By analyzing the user's social media activity, important information can be quickly grasped. Specifically, the monitoring unit receives as input post data obtained from social media APIs within the scope permitted by the user (e.g., post text, images, post time, location tags from the past week). The monitoring unit performs natural language analysis on post text using a large language model to extract keywords related to symptoms or conditions (e.g., “headache”, “fall”, “shortness of breath”) and emergency words (e.g., “help”, “ambulance”). Image data is processed by CNN to detect injuries or abnormal facial expressions, and post time and location tags are used for time-series and geographic context analysis. Generative AI integrates these features and outputs monitoring content (e.g., focused monitoring of headache and fall-related items, adjustment of monitoring frequency based on estimated urgency, prioritized input list). Examples of input include “post: ‘I've had a headache since this morning’”, “image: pale face”, “post time: 2024-06-01 08:00”. Examples of output include “priority monitoring items: headache, fatigue” and “estimated urgency: 0.7”. Based on this output, the monitoring unit automatically displays related items as input candidates on the monitoring screen, reducing the user's input burden. Furthermore, if urgency is high, the monitoring procedure can be simplified and switched to immediate reporting mode. As a technical effect, the monitoring unit realizes external information linkage, which was difficult with conventional user self-reporting or manual input, by combining AI-based social media analysis and optimization of monitoring content, thereby greatly improving monitoring efficiency, abnormality detection accuracy, and urgency determination accuracy. Application fields include emergency reporting apps, health management systems for young people, and mass health monitoring platforms during disasters. Thus, the monitoring unit achieves an improvement in computer technology as monitoring optimization through external data linkage, going beyond mere automation of human tasks.

The additional provision unit is capable of estimating the user's emotions and adjusting the method of providing additional information based on the estimated emotions. The additional provision unit uses generative AI to estimate the user's emotions and adjusts the method of providing additional information according to the estimated emotions. For example, if the user is nervous, the additional provision unit provides a simple and highly visible method for providing additional information. If the user is relaxed, the additional provision unit can provide detailed additional information. Furthermore, if the user is in a hurry, the additional provision unit can provide a method that focuses on key points for providing additional information. By adjusting the method of providing additional information according to the user's emotions, important information can be provided quickly. Emotion estimation is realized using an emotion estimation function, for example, by employing an emotion engine or generative AI. Generative AI may include text generation AI (e.g., LLM) or multimodal generative AI, but is not limited thereto. Specifically, the additional provision unit receives as input voice data obtained from the user terminal (e.g., 30 seconds of audio waveform sampled at 16 kHz), text data (e.g., natural language sentences such as “I want to know additional information”), image data (e.g., facial photos or expression images), and vital data (e.g., heart rate, skin conductance response). The additional provision unit performs spectrogram conversion and acoustic feature extraction (e.g., MFCC, pitch, energy) on the voice data, and extracts facial features from the image data using a convolutional neural network. The text data is processed by a large language model to estimate emotion labels (e.g., nervous, anxious, calm) and emotion scores (e.g., “nervous” with a score of 0.88). The vital data is analyzed by a time-series analysis model (e.g., LSTM) to calculate stress indices and abnormal values. These diverse features are input into a multimodal transformer-based integrated model, which outputs the final emotion estimation result (e.g., emotion category “nervous”, confidence 0.88). Examples of input include “voice: fast speech”, “text: ‘in a hurry’”, “image: furrowed brow”, “heart rate: 115 bpm”. Based on this emotion estimation result, the additional provision unit dynamically changes the method of providing additional information (e.g., UI layout, amount of information, highlighted items, color coding). For example, in a nervous state, only the most important items are prominently displayed, and detailed information is collapsed. In a relaxed state, detailed additional information, graphs, and historical comparison data are also displayed. In a hurry, only the key points are immediately displayed in a bulleted format. Examples of output include “display format: simple, highlighted item: additional severity”, “display format: detailed, with graph”, “display format: key points only”. These display controls are passed to the user interface generation module and used for automatic optimization of the terminal screen and notification content. As a technical effect, the additional provision unit can promptly respond to changes in user psychology, which was difficult with conventional uniform additional information display, and greatly improves information transmission efficiency, visibility, and user satisfaction by combining AI-based emotion estimation and display control. Application fields include emergency medical reporting apps, mass notification systems during disasters, elderly monitoring terminals, and remote health management for chronic disease patients. Thus, the additional provision unit achieves an improvement in computer technology as user state-linked additional information provision control, going beyond mere automation of human tasks.

The additional provision unit is capable of selecting an optimal method of providing additional information by referring to the user's past health data when providing additional information. The additional provision unit uses generative AI to select an optimal method of providing additional information by referring to the user's past health data when providing additional information. For example, the additional provision unit selects the optimal method of providing additional information based on the user's past health data. The additional provision unit can also prioritize the provision of important additional information by referring to the user's past health data. Furthermore, the additional provision unit can analyze the user's health data and select the optimal method of providing additional information. By referring to the user's past health data, the optimal method of providing additional information can be selected. Specifically, the additional provision unit obtains as input a health database accumulated for each user (e.g., time-series data for the past year such as heart rate, blood pressure, blood glucose, body temperature, medical history, medication history, past abnormality detection history). The additional provision unit preprocesses these data as time-series vectors and categorical features, and applies algorithms such as frequency analysis (e.g., frequent heart rate abnormalities in the past month), clustering (e.g., abnormal tendencies by symptom), and pattern extraction (e.g., blood pressure rises at specific times). Generative AI (e.g., large language models or time-series analysis models) recommends the optimal method of providing additional information for each user (e.g., focused information on heart rate, real-time notification of blood glucose, emphasis on body temperature information only at night), frequency of providing additional information, and priority of additional information items based on these features. Examples of input include “2024-06-01 14:23:10, heart rate 120 bpm, blood pressure 150/95 mmHg, medical history: hypertension” and “2024-05-20 08:10:00, blood glucose 180 mg/dL, medication: insulin”. Examples of output include “recommended method of providing additional information: heart rate every 1 minute, blood pressure every 10 minutes” and “priority additional information items: blood glucose, heart rate”. These outputs are reflected in the notification control module and user interface generation module of the additional provision unit, and a user-optimized additional information provision strategy is automatically executed. Furthermore, based on past abnormal occurrence patterns, preventive enhancement of additional information and automatic adjustment of alert thresholds are also possible. As a technical effect, the additional provision unit realizes user-specific optimization, which was difficult with conventional uniform methods of providing additional information, by combining AI-based history analysis and automatic optimization of additional information provision, thereby greatly improving information transmission efficiency, abnormality detection accuracy, and user satisfaction. Application fields include remote health management for chronic disease patients, employee health monitoring in companies, home medical support, and health monitoring during disasters. Thus, the additional provision unit achieves an improvement in computer technology as history-linked additional information provision optimization, going beyond mere automation of human tasks.

The additional provision unit can adjust the content of provision based on the user's current living conditions when providing additional information. The additional provision unit uses generative AI to adjust the content of provision based on the user's current living conditions at the time of providing additional information. For example, the additional provision unit adjusts the content of additional information provision in consideration of the user's current living conditions. Furthermore, the additional provision unit can prioritize the provision of important additional information based on the user's living conditions. Additionally, the additional provision unit can refer to the user's living conditions and propose optimal additional information provision content. By adjusting the content of provision based on the user's current living conditions, important information can be provided quickly. Specifically, the additional provision unit receives living condition data obtained from user terminals or wearable devices (e.g., step count and exercise intensity from activity trackers, sleep duration and sleep quality from sleep sensors, meal content from meal recording apps, outing schedules from calendar integration) as input. The additional provision unit preprocesses these data as time-series vectors or categorical features and applies algorithms such as living pattern analysis (e.g., high activity during the day, sleep at night), abnormal behavior detection (e.g., sudden decrease in activity, sleep deprivation), and life event detection (e.g., travel, long outings). Generative AI (e.g., large language models or time-series analysis models) recommends additional information provision content optimized for the user's current living conditions (e.g., prioritizing heart rate and fall detection information during exercise, providing respiration and heart rate variability information during sleep, enhancing location and fall detection information during outings) and the priority of provision items based on these features. Examples of input include “Activity: 10,000 steps, Sleep duration: 6 hours, Outing schedule: 14:00-18:00” and “Meal content: high salt, Exercise intensity: high.” Examples of output include “Priority additional information items: heart rate, fall detection (during exercise)” and “Additional information content: respiration, heart rate variability (during sleep).” These outputs are reflected in the notification control module and user interface generation module of the additional provision unit, enabling dynamic additional information provision strategies according to living conditions to be automatically executed. As a technical effect, the additional provision unit realizes rapid response to changes in living conditions, which was difficult with conventional uniform additional information content settings, by combining AI-based living data analysis and automatic optimization of additional information content, thereby greatly improving information transmission efficiency, abnormality detection accuracy, and user satisfaction. Application fields include elderly monitoring, home medical care, health management for patients with chronic diseases, and corporate health monitoring. Thus, the additional provision unit achieves an improvement in computer technology as a living condition-linked additional information provision optimization, going beyond mere automation of human tasks.

The additional provision unit can estimate the user's emotions and determine the priority of additional information based on the estimated emotions. The additional provision unit uses generative AI to estimate the user's emotions and determine the priority of additional information based on the estimated emotions. For example, if the user is tense, the additional provision unit prioritizes the provision of important additional information. If the user is relaxed, the additional provision unit can provide detailed additional information. Furthermore, if the user is in a hurry, the additional provision unit can quickly provide only the most important additional information. By determining the priority of additional information according to the user's emotions, important information can be provided quickly. Emotion estimation is realized using emotion engines or generative AI, such as text generation AI (e.g., LLM) or multimodal generative AI, but is not limited to these examples. Specifically, the additional provision unit receives voice data, text data, image data, and vital data obtained from the user as input. The additional provision unit extracts acoustic features (e.g., MFCC, pitch) from voice data, facial features using convolutional neural networks from image data, and emotion classification (e.g., tension, relaxation, urgency) using large language models from text data. Vital data are analyzed by time-series analysis models to calculate stress indices. These features are input into a multimodal transformer, which outputs emotion categories (e.g., tension, relaxation, urgency) and confidence scores. Examples of output include “Emotion: tension, confidence 0.88” and “Emotion: relaxation, confidence 0.72.” Based on these emotion estimation results, the additional provision unit automatically determines the priority of additional information (e.g., most important items→key points→detailed information) and dynamically controls the order of notifications and screen display. For example, in a tense state, the most important items such as “additional severity” and “urgency” are displayed first, while in a relaxed state, detailed items such as “medical history” and “medication information” are displayed sequentially. In urgent cases, only the minimum required items are displayed immediately. These priority information are passed to the user interface generation module and notification control module and used for dynamic optimization of additional information screens and notification content. As a technical effect, the additional provision unit responds quickly to psychological changes in emergencies, which was difficult with conventional uniform additional information order, and greatly improves information transmission efficiency, speed, and user satisfaction by combining AI-based emotion estimation and priority control. Application fields include emergency medical reporting apps, mass notification systems during disasters, elderly monitoring devices, and remote health management for patients with chronic diseases. Thus, the additional provision unit achieves an improvement in computer technology as a user state-linked additional information priority control, going beyond mere automation of human tasks.

The additional provision unit can select the optimal provision method by considering the user's geographic location information when providing additional information. The additional provision unit uses generative AI to select the optimal provision method by considering the user's geographic location information at the time of providing additional information. For example, the additional provision unit selects the optimal additional information provision method based on the user's geographic location information. Furthermore, the additional provision unit can prioritize the provision of geographically relevant information. Additionally, the additional provision unit can refer to the user's geographic location information and propose the optimal additional information provision method. By considering the user's geographic location information, the optimal additional information provision method can be selected. Specifically, the additional provision unit receives latitude and longitude data (e.g., 35.6895, 139.6917) obtained from the GPS sensor of the user terminal or network location information API as input. The additional provision unit collaborates with a geographic information system (GIS) database to obtain external data such as lists of nearby medical institutions, information on local infectious disease outbreaks, and disaster occurrence status. Generative AI (e.g., large language models or geographic information linkage modules) combines location information and external data to recommend additional information provision methods (e.g., prioritizing body temperature and respiration information in infectious disease outbreak areas, enhancing fall and trauma information in disaster areas, emphasizing location and battery status information in remote areas) and the priority of provision items. Examples of input include “Location: 35.6895, 139.6917, Disaster: earthquake occurred” and “Location: 34.6937, 135.5023, Infectious disease outbreak: influenza.” Examples of output include “Recommended additional information provision method: focus on body temperature and respiration (infectious disease outbreak area)” and “Priority additional information items: fall detection, trauma (disaster area).” These outputs are reflected in the notification control module and user interface generation module of the additional provision unit, enabling dynamic additional information provision strategies according to geographic conditions to be automatically executed. As a technical effect, the additional provision unit realizes rapid response to regional characteristics, which was difficult with conventional uniform additional information provision method settings, by combining AI-based location information analysis and automatic optimization of additional information provision methods, thereby greatly improving information transmission efficiency, abnormality detection accuracy, and regional medical cooperation. Application fields include emergency reporting apps, mass notification systems during disasters, health management apps for travelers, and remote health monitoring. Thus, the additional provision unit achieves an improvement in computer technology as a geographic information-linked additional information provision optimization, going beyond mere automation of human tasks.

The additional provision unit can analyze the user's social media activity and adjust the content of provision when providing additional information. The additional provision unit uses generative AI to analyze the user's social media activity and adjust the content of provision at the time of providing additional information. For example, the additional provision unit extracts information related to the current situation or symptoms from the user's social media posts and adjusts the content of additional information provision. Furthermore, the additional provision unit can analyze the user's social media activity and prioritize the provision of additional information related to emergencies. Additionally, the additional provision unit can refer to the user's social media data and propose optimal additional information provision content. By analyzing the user's social media activity, important information can be provided quickly. Specifically, the additional provision unit receives post data obtained from social media APIs within the scope permitted by the user (e.g., post text, images, posting time, location tags from the past week) as input. The additional provision unit performs natural language analysis using large language models on post text to extract keywords related to symptoms and situations (e.g., “headache,” “fall,” “shortness of breath”) and emergency words (e.g., “help,” “ambulance”). Image data are analyzed by CNN to detect trauma or abnormal facial expressions, and posting time and location tags are used for time-series and geographic context analysis. Generative AI integrates these features and outputs additional information provision content (e.g., focus on headache and fall-related items, notification frequency adjustment based on estimated urgency, additional information priority list). Examples of input include “Post: ‘I've had a headache since this morning,’” “Image: pale face,” “Posting time: 2024-06-01 08:00.” Examples of output include “Priority additional information items: headache, fatigue” and “Estimated urgency: 0.7.” Based on these outputs, the additional provision unit automatically displays related items as auto-completion candidates on the notification screen, reducing the user's input burden. Furthermore, if urgency is high, the notification procedure can be simplified and switched to immediate reporting mode. As a technical effect, the additional provision unit realizes external information linkage, which was difficult with conventional user self-reporting or manual input, by combining AI-based social media analysis and optimization of additional information content, thereby greatly improving information transmission efficiency, abnormality detection accuracy, and urgency determination accuracy. Application fields include emergency reporting apps, health management systems for young people, and mass notification platforms during disasters. Thus, the additional provision unit achieves an improvement in computer technology as an external data-linked additional information provision optimization, going beyond mere automation of human tasks.

The system according to the embodiment is not limited to the examples described above and can be variously modified as follows, for example. Specifically, the system can flexibly expand or reduce the functions of each component, such as the reception unit, analysis unit, provision unit, decision unit, monitoring unit, and additional provision unit, according to the user's usage environment or requirements of the medical field. The system can be applied to both a centralized configuration in which all functions are integrated and implemented on a single server, and a distributed configuration in which functions are executed in a cloud environment or on edge devices. Furthermore, the type and training method of AI models can also be changed; for example, the speech recognition unit can select convolutional neural networks, recurrent neural networks, or Transformer-based speech models. The emotion estimation unit can use a combination of multimodal integration models, large language models, and time-series analysis models. Regarding data flow, the optimal method can be selected according to system requirements, such as real-time streaming processing, batch processing, or event-driven processing. The types of input data can also be integrated from various data sources, including voice, text, images, vital data, location information, social media data, and external data from medical institutions. As for AI model training methods, supervised learning, semi-supervised learning, transfer learning, online learning, and reinforcement learning can be applied as appropriate. Furthermore, the algorithms of the analysis unit and decision unit can implement hybrid methods combining rule-based processing and AI inference, or accuracy improvement methods using ensemble of multiple AI models. The output format of the system can also support various formats, such as structured data, natural language generation, graph display, alert notification, and API integration. As a technical effect, the system realizes flexible functional expandability, operational adaptability, and optimal selection of AI models, which were difficult to achieve with conventional fixed medical information systems, and can respond quickly to diverse needs in medical and disaster response fields. Application fields include emergency medical reporting apps, remote diagnostic support, mass notification platforms during disasters, remote health management for patients with chronic diseases, and corporate health monitoring systems. Thus, the system achieves a fundamental improvement in computer technology as an AI and data-linked medical information processing platform, going beyond mere automation of human tasks.

The reception unit can refer to the user's past health data and automatically complete input content when receiving input of the user's symptoms and conditions. For example, if the user has a specific medical history, the reception unit automatically presents symptoms related to that medical history as input candidates. If the user has taken a specific medication in the past, the reception unit can automatically include information about that medication in the input content. Furthermore, based on the user's past health data, the reception unit can automatically provide information about relevant medical institutions. By utilizing the user's past health data, rapid and accurate information input becomes possible. Specifically, the reception unit obtains a health database accumulated for each user (e.g., structured data such as medical history for the past year, medication history, past medical history, test values, allergy information) as input. The reception unit simultaneously inputs the symptom/condition text currently being entered by the user (e.g., “chest discomfort”) or selection options and past health data vectors (e.g., medical history: hypertension, medication: antihypertensive drugs, past symptoms: headache, fever) into an input completion AI module (e.g., large language model or rule-based completion engine). The AI module calculates the relevance score between past data and current input content and generates highly relevant symptoms, drug names, and medical institution names as auto-completion candidates. Examples of input include “Current input: chest discomfort, past medical history: myocardial infarction” and “Current input: fever, past medication: antibiotics.” Examples of output include “Completion candidate: history of myocardial infarction→chest pain, shortness of breath” and “Completion candidate: history of antibiotic use→check for allergies.” The reception unit automatically displays these completion candidates on the user interface so that the user can select them with a single tap. Furthermore, information about relevant medical institutions (e.g., hospitals previously visited, list of specialized departments) is also automatically presented to reduce input burden. For AI model training, supervised learning using past input history and correct completion results, and online learning reflecting user selection tendencies can be applied. As a technical effect, the reception unit realizes user-specific optimization, input efficiency, and reduction of input errors, which were difficult with conventional manual input or static candidate presentation, by combining AI-based history-referenced auto-completion, thereby greatly improving the speed, accuracy, and user satisfaction of information input. Application fields include emergency medical reporting apps, health management apps for patients with chronic diseases, and corporate health monitoring systems. Thus, the reception unit achieves an improvement in computer technology as a history-linked input completion, going beyond mere automation of human tasks.

The monitoring unit can adjust the timing of monitoring based on the user's daily rhythm when continuously monitoring the user's symptoms and conditions. For example, if the user is sleeping at night, the monitoring unit reduces the monitoring frequency at night and increases the monitoring frequency during daytime activity hours. If the user exercises regularly, the monitoring unit can perform monitoring according to the exercise schedule. Furthermore, the monitoring unit can notify monitoring results at appropriate times based on the user's daily rhythm. By monitoring according to the user's daily rhythm, more effective health management can be achieved. Specifically, the monitoring unit receives daily rhythm data obtained from user terminals or wearable devices (e.g., step count and exercise intensity from activity trackers, sleep duration and sleep quality from sleep sensors, schedule information from calendar integration, meal times from meal recording apps) as input. The monitoring unit preprocesses these data as time-series vectors or categorical features and uses daily pattern analysis AI (e.g., time-series clustering models, LSTM, autoregressive models) to automatically extract rhythm intervals such as “activity hours,” “sleep hours,” and “exercise hours.” Examples of input include “Activity: 10,000 steps, Sleep duration: 6 hours, Exercise schedule: 18:00-19:00.” The monitoring unit dynamically adjusts the parameters of the monitoring frequency control module for each extracted daily rhythm interval (e.g., 1-minute intervals during activity, 30-minute intervals during sleep, focused monitoring of heart rate and fall detection during exercise). Furthermore, the timing of notification of monitoring results is optimized according to the user's daily rhythm, such as suppressing notifications during sleep and summarizing notifications after waking up or at the start of activity. Examples of output include “Monitoring frequency: 1-minute intervals (during activity)” and “Notification timing: batch notification after waking up.” These controls are reflected in the sensor data acquisition module and notification control module, enabling automatic execution of monitoring strategies according to the user's daily rhythm. As a technical effect, the monitoring unit realizes rapid response to changes in daily rhythm, which was difficult with conventional uniform monitoring timing settings, by combining AI-based daily pattern analysis and automatic timing optimization, thereby greatly improving monitoring efficiency, abnormality detection accuracy, and user satisfaction. Application fields include elderly monitoring, home medical care, health management for patients with chronic diseases, and corporate health monitoring. Thus, the monitoring unit achieves an improvement in computer technology as a daily rhythm-linked monitoring timing optimization, going beyond mere automation of human tasks.

The additional provision unit can provide additional information to the nearest medical institution or emergency call receiving agency based on the user's current location information when the user's symptoms worsen. For example, if the user is in a specific area, the additional provision unit provides information about the medical institution closest to that area. Furthermore, based on the user's location information, the additional provision unit can propose optimal deployment of ambulances and transport routes. Additionally, by updating the user's location information in real time and providing it to emergency call receiving agencies or medical institutions, rapid response becomes possible. By utilizing the user's location information, more effective rescue activities can be realized. Specifically, the additional provision unit receives latitude and longitude data (e.g., 35.6895, 139.6917) obtained from the GPS sensor of the user terminal or network location information API as input. The additional provision unit collaborates with a geographic information system (GIS) database to obtain external data such as lists of nearby medical institutions, ambulance deployment bases, and disaster area information. The AI module (e.g., large language model or geographic information linkage module) combines location information and external data to automatically calculate the selection of the nearest medical institution, optimal ambulance deployment points, and transport routes (e.g., shortest distance route, congestion avoidance route, safe route during disasters). Examples of input include “User location: 35.6895, 139.6917, symptoms: worsening” and “Disaster area: 35.6900, 139.7000.” Examples of output include “Additional information provision destination: Hospital A (distance 0.8 km)” and “Recommended deployment: ambulance ID123, route A→B.” The additional provision unit automatically transmits this information in real time to emergency call receiving agencies or medical institutions, where it is used for rapid decision-making and rescue preparation on site. Furthermore, as the user moves, location information is continuously updated, and transport routes and deployment instructions can be dynamically recalculated. As a technical effect, the additional provision unit realizes real-time location-linked additional information provision, which was difficult with conventional static information provision or manual judgment by human operators, by combining AI-based location information analysis and automated decision-making, thereby greatly improving the speed of rescue activities, on-site responsiveness, and transport efficiency. Application fields include emergency medical care in urban and rural areas, wide-area transport during disasters, remote medical support, and corporate safety management systems. Thus, the additional provision unit achieves an improvement in computer technology as a location information-linked additional information provision optimization, going beyond mere automation of human tasks.

The analysis unit can analyze the user's social media activity to obtain additional information when analyzing the user's symptoms and conditions. For example, the analysis unit extracts information related to current symptoms and conditions from the user's social media posts. Furthermore, the analysis unit can analyze the user's social media activity and prioritize the analysis of information related to emergencies. Additionally, the analysis unit can refer to the user's social media data and provide additional information to supplement the analysis results. By utilizing the user's social media activity, more accurate analysis becomes possible. Specifically, the analysis unit receives post data obtained from social media APIs within the scope permitted by the user (e.g., post text, images, posting time, location tags from the past week) as input. The analysis unit performs natural language analysis using large language models on post text to extract keywords related to symptoms and conditions (e.g., “headache,” “fall,” “shortness of breath”) and emergency words (e.g., “help,” “ambulance”). Image data are analyzed by convolutional neural networks to detect trauma or abnormal facial expressions, and posting time and location tags are used for time-series and geographic context analysis. The AI module integrates these features and outputs analysis content (e.g., focus on headache and fall-related items, determination of analysis priority based on estimated urgency, list of input completion candidates). Examples of input include “Post: ‘I've had a headache since this morning,’” “Image: pale face,” “Posting time: 2024-06-01 08:00.” Examples of output include “Priority analysis items: headache, fatigue” and “Estimated urgency: 0.7.” Based on these outputs, the analysis unit automatically displays related items as auto-completion candidates on the analysis screen, reducing the user's input burden. Furthermore, if urgency is high, the analysis procedure can be simplified and switched to immediate reporting mode. As a technical effect, the analysis unit realizes external information linkage, which was difficult with conventional user self-reporting or manual input, by combining AI-based social media analysis and optimization of analysis content, thereby greatly improving analysis efficiency, abnormality detection accuracy, and urgency determination accuracy. Application fields include emergency reporting apps, health management systems for young people, and mass health monitoring platforms during disasters. Thus, the analysis unit achieves an improvement in computer technology as an external data-linked analysis optimization, going beyond mere automation of human tasks.

The provision unit can estimate the user's emotions and adjust the method of information provision based on the estimated emotions when providing detailed information to emergency call receiving agencies or medical institutions. For example, if the user is tense, the provision unit selects a simple and highly visible information provision method. If the user is relaxed, the provision unit can select a provision method that includes detailed information. Furthermore, if the user is in a hurry, the provision unit can select a method that provides only the key points. By adjusting the method of information provision according to the user's emotions, important information can be provided quickly. Specifically, the provision unit receives voice data (e.g., 30-second audio waveform sampled at 16 kHz), text data (e.g., natural language sentences such as “difficulty breathing”), image data (e.g., facial photos or expression images), and vital data (e.g., heart rate, skin conductance) obtained from the user as input. The provision unit performs spectrogram conversion and acoustic feature extraction (e.g., MFCC, pitch, energy) on voice data, and facial feature extraction using convolutional neural networks on image data. Text data are analyzed by large language models to estimate emotion labels (e.g., tension, anxiety, calmness) and emotion scores (e.g., 0.88 for “tension”). Vital data are analyzed by time-series analysis models (e.g., LSTM) to calculate stress indices and abnormal values. These diverse features are input into a multimodal transformer (Transformer-based integration model), which outputs the final emotion estimation result (e.g., emotion category “tension,” confidence 0.88). Examples of input include “Voice: trembling voice,” “Text: ‘difficulty breathing,’” “Image: tense face,” “Heart rate: 110 bpm.” Based on this emotion estimation result, the provision unit dynamically changes the information provision method (e.g., UI layout, amount of information, highlighted items, color coding). For example, in a tense state, only the most important items are prominently displayed and detailed information is collapsed. In a relaxed state, detailed analysis results, graphs, and past comparison data are also displayed. In urgent cases, only the key points are displayed immediately in a bulleted list. Examples of output include “Display format: simple, highlighted item: severity,” “Display format: detailed, with graph,” and “Display format: key points only.” These display controls are passed to the user interface generation module and used for automatic optimization of terminal screens and notification content. As a technical effect, the provision unit responds quickly to psychological changes in users, which was difficult with conventional uniform display methods, and greatly improves information transmission efficiency, visibility, and user satisfaction by combining AI-based emotion estimation and display control. Application fields include emergency medical reporting apps, mass notification systems during disasters, and elderly monitoring devices. Thus, the provision unit achieves an improvement in computer technology as a user state-linked display control, going beyond mere automation of human tasks.

The decision unit can monitor current traffic conditions in real time and select the optimal route when determining the deployment of emergency vehicles or transport routes. For example, the decision unit selects the fastest route based on traffic congestion information. It can also propose detour routes by considering information such as traffic accidents and road construction. Furthermore, the decision unit can recalculate routes in real time according to changes in traffic conditions and provide the optimal transport route. By considering current traffic conditions, rapid transport becomes possible. Specifically, the decision unit receives real-time traffic condition data (e.g., congestion level for each road, accident locations, construction sections, estimated travel time, traffic regulation information) obtained from traffic information APIs or geographic information systems as input. The decision unit preprocesses these traffic data as graph structures (e.g., road network nodes and edges, each edge assigned travel cost or congestion score) and applies shortest path search algorithms (e.g., Dijkstra's algorithm, A* algorithm) and real-time route recalculation algorithms. The AI module (e.g., large language model or reinforcement learning model) also considers traffic pattern changes and past transport records to output optimal deployment and transport routes (e.g., congestion avoidance route, accident detour route, simultaneous deployment patterns for multiple vehicles). Examples of input include “Point A to Hospital B: normally 15 minutes, currently congested +10 minutes” and “Road C: accident occurred, closed.” Examples of output include “Recommended route: A→D→B (congestion avoidance, required time 18 minutes)” and “Deployment instruction: vehicle ID789, departure time 14:28, use detour route.” These outputs are reflected in the deployment and transport algorithms of the decision unit, automating optimal decision-making according to on-site conditions. Furthermore, the decision unit monitors changes in traffic conditions in real time during transport and can immediately recalculate routes or change deployments as needed. As a technical effect, the decision unit realizes real-time traffic-linked optimization, which was difficult with conventional static route selection or manual judgment by human operators, by combining AI-based traffic data analysis and automated decision-making, thereby greatly improving transport efficiency, speed, and on-site responsiveness. Application fields include emergency medical care in urban areas, wide-area transport during disasters, remote medical support, and corporate safety management systems. Thus, the decision unit achieves an improvement in computer technology as a traffic information-linked deployment and transport optimization, going beyond mere automation of human tasks.

The reception unit can estimate the user's emotions and adjust the input method for symptoms and conditions based on the estimated emotions. For example, if the user is in a panic state, the reception unit provides a simple and intuitive interface and minimizes the input steps. If the user is calm, the reception unit provides detailed input options and proposes customizable input methods. Furthermore, if the user is facing an emergency, the reception unit prioritizes voice input to enable rapid input of symptoms and conditions. By adjusting the input method according to the user's emotions, more appropriate information can be entered quickly. Specifically, the reception unit receives voice data (e.g., 30-second audio waveform sampled at 16 kHz), text data (e.g., natural language sentences such as “help”), image data (e.g., facial photos or expression images), and vital data (e.g., heart rate, skin conductance) obtained from the user as input. The reception unit performs spectrogram conversion and acoustic feature extraction (e.g., MFCC, pitch, energy) on voice data, and facial feature extraction using convolutional neural networks on image data. Text data are analyzed by large language models to estimate emotion labels (e.g., panic, calm, emergency) and emotion scores (e.g., 0.92 for “panic”). Vital data are analyzed by time-series analysis models (e.g., LSTM) to calculate stress indices and abnormal values. These diverse features are input into a multimodal transformer (Transformer-based integration model), which outputs the final emotion estimation result (e.g., emotion category “panic,” confidence 0.92). Examples of input include “Voice: fast speech,” “Text: ‘help,’” “Image: tense face,” “Heart rate: 120 bpm.” Based on this emotion estimation result, the reception unit dynamically changes the parameters of the input interface control module (e.g., UI layout, number of input steps, input method priority). For example, in a panic state, only the minimum required items are prominently displayed and voice input is automatically activated. In a calm state, detailed input options and customization items are displayed sequentially. In emergencies, voice and image input are prioritized to enable rapid information acquisition. Examples of output include “Input method: voice prioritized, number of items: 3 (panic state)” and “Input method: detailed options, number of items: 10 (calm state).” These controls are reflected in the user interface generation module and used for automatic optimization of terminal screens and input procedures. As a technical effect, the reception unit responds quickly to psychological changes and urgency fluctuations, which were difficult with conventional uniform input methods, and greatly improves information input efficiency, accuracy, and user satisfaction by combining AI-based emotion estimation and input method control. Application fields include emergency medical reporting apps, mass notification systems during disasters, and elderly monitoring devices. Thus, the reception unit achieves an improvement in computer technology as a user state-linked input method control, going beyond mere automation of human tasks.

The reception unit can analyze the user's past emergency call history and provide an appropriate input interface. For example, the reception unit prioritizes input methods (voice, text, etc.) that the user has frequently used in the past. Furthermore, based on the user's past emergency call history, the reception unit can automatically display input options corresponding to specific symptoms or conditions. Additionally, the reception unit can analyze the user's past emergency call history and propose input interfaces used during specific time periods. By analyzing the user's past emergency call history, the optimal input interface can be provided. Specifically, the reception unit obtains an emergency call history database accumulated for each user (e.g., structured data such as call date and time for the past year, input method, input content, symptoms/conditions, call success rate) as input. The reception unit inputs features such as frequency of past input method usage, input tendencies for symptoms/conditions, and usage patterns by time period into a history analysis AI module (e.g., large language model or time-series analysis model). The AI module uses these features to automatically generate the optimal input interface (e.g., voice input, text input, image input, selection input) and input options (e.g., frequently used symptom candidates, situation selection options) for the current reporting situation. Examples of input include “Past call: 2024-06-01 14:23:10, voice input, symptom: chest pain” and “Past call: 2024-05-20 08:10:00, text input, symptom: fever.” Examples of output include “Recommended input method: voice input (usage frequency 80%)” and “Recommended input options: chest pain, fever.” The reception unit automatically displays these recommended interfaces and options on the user interface so that the user can select them with a single tap. Furthermore, usage tendencies by time period are reflected, enabling dynamic control such as prioritizing voice input at night and text input during the day. As a technical effect, the reception unit realizes user-specific optimization, input efficiency, and reduction of input errors, which were difficult with conventional static input method presentation or manual selection, by combining AI-based history analysis interface optimization, thereby greatly improving the speed, accuracy, and user satisfaction of information input. Application fields include emergency medical reporting apps, health management apps for patients with chronic diseases, and corporate health monitoring systems. Thus, the reception unit achieves an improvement in computer technology as a history-linked input interface optimization, going beyond mere automation of human tasks.

The reception unit can filter input content for symptoms and conditions based on the user's current health status and medical history at the time of input. For example, the reception unit prioritizes the input of symptoms and conditions related to the user's medical history. Furthermore, the reception unit can automatically filter input content by considering the user's current health status and emphasize important information. Additionally, the reception unit can refer to the user's health data and perform filtering to optimize input content. By filtering input content based on the user's current health status and medical history, important information can be emphasized. Specifically, the reception unit obtains a health database accumulated for each user (e.g., structured data such as recent vital data, medical history, medication history, test values, allergy information) as input. The reception unit simultaneously inputs the symptom/condition text or selection options currently being entered and health data vectors into an input filtering AI module (e.g., large language model or rule-based filtering engine). The AI module calculates the relevance score between medical history/current health status and input content, and displays highly relevant symptoms and conditions as prioritized input candidates, while emphasizing important information (e.g., highly severe symptoms, symptoms related to medical history). Examples of input include “Medical history: hypertension, current input: headache” and “Vital: heart rate 120 bpm, input candidate: palpitations.” Examples of output include “Priority input candidates: headache, palpitations (related to medical history)” and “Highlighted item: high severity.” The reception unit automatically reflects these filtering results on the user interface so that the user can input important items without overlooking them. Furthermore, input candidates and highlighted items can be dynamically updated according to changes in health status. As a technical effect, the reception unit realizes user-specific optimization, input efficiency, and emphasis of important information, which were difficult with conventional static input candidate presentation or manual selection, by combining AI-based health data-linked input filtering, thereby greatly improving the accuracy, speed, and user satisfaction of information input. Application fields include emergency medical reporting apps, health management apps for patients with chronic diseases, and corporate health monitoring systems. Thus, the reception unit achieves an improvement in computer technology as a health data-linked input filtering, going beyond mere automation of human tasks.

The reception unit can estimate the user's emotions and determine the priority of input content based on the estimated emotions. For example, if the user is tense, the reception unit prioritizes the input of important information. If the user is relaxed, the reception unit can prompt the input of detailed information. Furthermore, if the user is in a hurry, the reception unit can prompt the rapid input of only the most important information. By determining the priority of input content according to the user's emotions, important information can be entered quickly. Specifically, the reception unit receives voice data, text data, image data, and vital data obtained from the user as input. The reception unit extracts acoustic features (e.g., MFCC, pitch) from voice data, facial features using convolutional neural networks from image data, and emotion classification (e.g., tension, relaxation, urgency) using large language models from text data. Vital data are analyzed by time-series analysis models to calculate stress indices. These features are input into a multimodal transformer, which outputs emotion categories (e.g., tension, relaxation, urgency) and confidence scores. Examples of output include “Emotion: tension, confidence 0.88” and “Emotion: relaxation, confidence 0.72.” Based on these emotion estimation results, the reception unit automatically determines the priority of input content (e.g., most important items→key points→detailed information) and dynamically controls the display order of input screens and input procedures. For example, in a tense state, the most important items such as “severity” and “urgency” are displayed first, while in a relaxed state, detailed items such as “medical history” and “medication information” are displayed sequentially. In urgent cases, only the minimum required items are displayed immediately. These priority information are passed to the user interface generation module and used for dynamic optimization of input screens and notification content. As a technical effect, the reception unit responds quickly to psychological changes in emergencies, which was difficult with conventional uniform input order, and greatly improves information input efficiency, speed, and user satisfaction by combining AI-based emotion estimation and priority control. Application fields include emergency medical reporting apps, mass notification systems during disasters, and elderly monitoring devices. Thus, the reception unit achieves an improvement in computer technology as a user state-linked input priority control, going beyond mere automation of human tasks.

The following is a brief explanation of the processing flow of Example of the Embodiment. Specifically, the system operates in cooperation among multiple functional modules such as the reception unit, analysis unit, provision unit, decision unit, monitoring unit, and additional provision unit. First, the reception unit receives various input data from the user terminal, including voice data, text data, image data, vital data, location information, and social media data. The reception unit executes AI-based emotion estimation, history analysis, health data-linked input completion, filtering, and priority control, and automatically generates a user-optimized input interface. Next, the analysis unit analyzes the data received from the reception unit using multimodal AI models (e.g., large language models, convolutional neural networks, time-series analysis models, Transformer-based integration models) to comprehensively analyze symptoms, conditions, emotions, urgency, relevance, and occurrence time, and generates analysis results. The analysis unit also refers to social media data and past history to improve analysis accuracy through external information linkage. The provision unit optimizes information provision content, display method, priority, and notification method using AI, based on the results of the analysis unit and considering the category, importance, geographic location, and related literature of emergency call receiving agencies or medical institutions. The decision unit integrates information from the provision unit with real-time traffic information, past deployment history, geographic information, and related literature, and executes AI-based optimization of emergency vehicle deployment, transport routes, priority determination, and automated decision-making. The monitoring unit analyzes the user's daily rhythm, health status, emotions, geographic information, and social media activity using AI, and optimizes monitoring frequency, content, priority, and method. The additional provision unit utilizes AI-based history analysis, living condition analysis, geographic information linkage, and external data linkage to automatically determine optimal additional information provision content, method, priority, and notification strategy when symptoms worsen or additional information is requested. These units cooperate in terms of AI model input/output specifications, data flow, and algorithm control, greatly improving individual optimization, speed, accuracy, and on-site responsiveness, which were difficult to achieve with conventional uniform medical information processing. Application fields include emergency medical reporting apps, mass notification systems during disasters, remote diagnostic support, elderly monitoring devices, remote health management for patients with chronic diseases, and corporate health monitoring systems. Thus, the system achieves a fundamental improvement in computer technology as an AI and data-linked medical information processing platform, going beyond mere automation of human tasks.

Step 1: The reception unit receives input of the user's symptoms and conditions. The user's symptoms and conditions include health status, emergencies, accidents, and so on. The reception unit can receive input from the user via a smartphone application and supports multiple input methods such as voice input and text input. Step 2: The analysis unit uses generative AI to analyze the information received by the reception unit and determine the user's symptoms and conditions. The analysis is performed based on data analysis algorithms and diagnostic criteria to determine the severity and urgency of symptoms. The analysis unit also continuously monitors the user's symptoms and conditions and provides additional information as needed. Step 3: The provision unit uses generative AI to provide information determined by the analysis unit to an emergency call receiving agency or a medical institution. The provision is performed based on communication means and information format, and transmits detailed information about the user's symptoms and conditions. Step 4: The decision unit uses generative AI to determine deployment of emergency vehicles or a transport route based on the information provided by the provision unit. The decision is made based on urgency evaluation criteria and route selection algorithms to determine deployment of emergency vehicles and the optimal transport route, and arrange rapid rescue. Specifically, in Step 1, the reception unit receives various input data from the user terminal, including voice data (e.g., 30-second audio waveform sampled at 16 kHz), text data (e.g., natural language sentences such as “difficulty breathing”), image data (e.g., facial photos or expression images), vital data (e.g., heart rate, skin conductance), location information, and social media data. The reception unit executes AI-based emotion estimation, history analysis, health data-linked input completion, filtering, and priority control, and automatically generates a user-optimized input interface. In Step 2, the analysis unit analyzes the data received from the reception unit using multimodal AI models (e.g., large language models, convolutional neural networks, time-series analysis models, Transformer-based integration models) to comprehensively analyze symptoms, conditions, emotions, urgency, relevance, and occurrence time, and generates analysis results. The analysis unit also refers to social media data and past history to improve analysis accuracy through external information linkage. In Step 3, the provision unit optimizes information provision content, display method, priority, and notification method using AI, based on the results of the analysis unit and considering the category, importance, geographic location, and related literature of emergency call receiving agencies or medical institutions. In Step 4, the decision unit integrates information from the provision unit with real-time traffic information, past deployment history, geographic information, and related literature, and executes AI-based optimization of emergency vehicle deployment, transport routes, priority determination, and automated decision-making. These units cooperate in terms of AI model input/output specifications, data flow, and algorithm control, greatly improving individual optimization, speed, accuracy, and on-site responsiveness, which were difficult to achieve with conventional uniform medical information processing. Application fields include emergency medical reporting apps, mass notification systems during disasters, remote diagnostic support, elderly monitoring devices, remote health management for patients with chronic diseases, and corporate health monitoring systems. Thus, the system achieves a fundamental improvement in computer technology as an AI and data-linked medical information processing platform, going beyond mere automation of human tasks.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-mentioned elements, including the reception unit, analysis unit, provision unit, decision unit, monitoring unit, and additional provision unit, is implemented by at least one of, for example, the smart device 14 and the data processing apparatus 12. For example, the reception unit receives input from the user via an application of the smart device 14. The analysis unit is implemented by a specific processing unit 290 of the data processing apparatus 12 and analyzes the user's symptoms and conditions using generative AI. The provision unit is implemented by the specific processing unit 290 of the data processing apparatus 12 and provides the analyzed information to an emergency call receiving agency or a medical institution. The decision unit is implemented by the specific processing unit 290 of the data processing apparatus 12 and determines deployment of emergency vehicles or a transport route. The monitoring unit is implemented by a control unit 46A of the smart device 14 and continuously monitors the user's symptoms and conditions. The additional provision unit is implemented by the specific processing unit 290 of the data processing apparatus 12 and provides additional information when the user's symptoms worsen. The correspondence between each unit and the apparatus or control unit is not limited to the examples described above and various modifications are possible.

### Second Embodiment

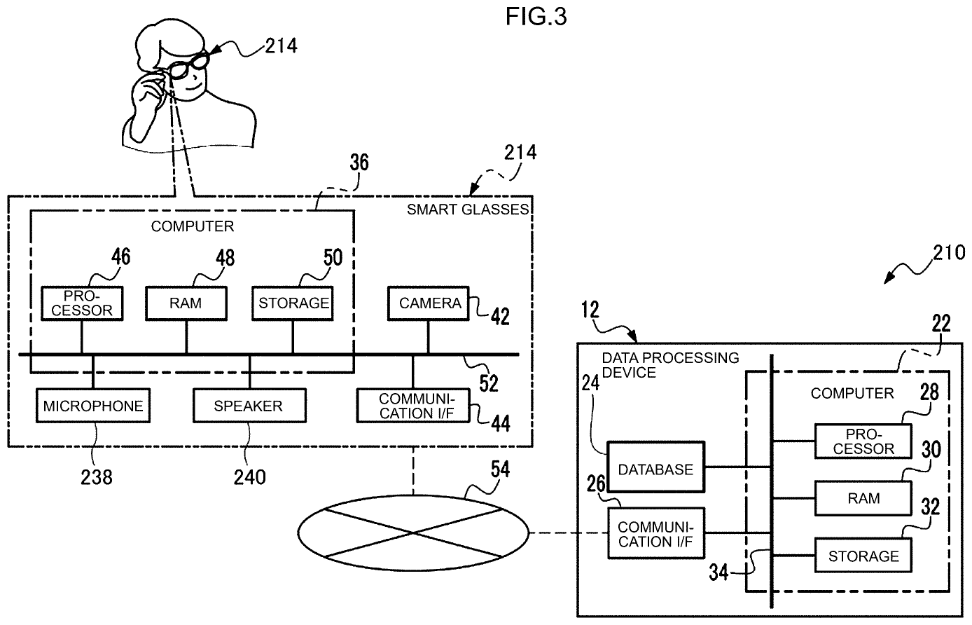

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

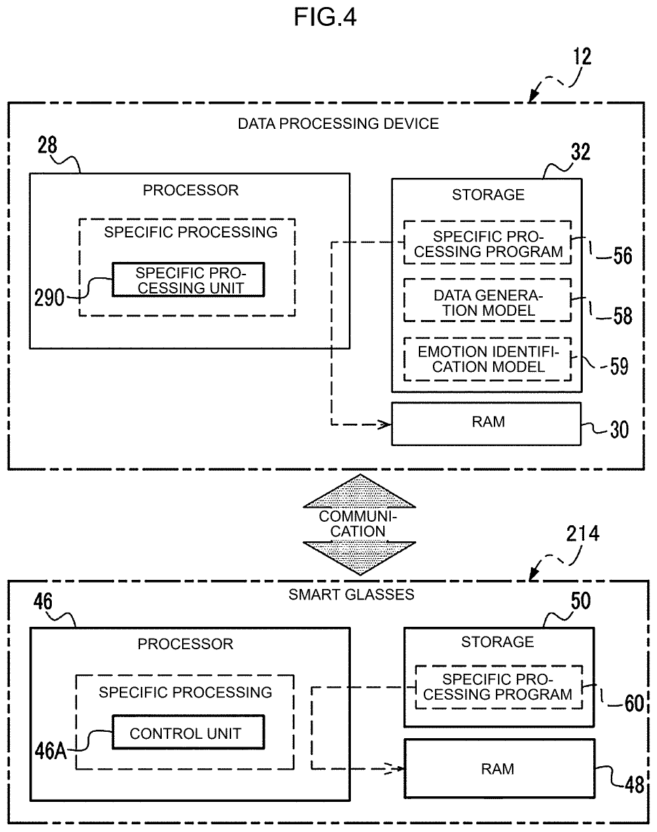

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-mentioned elements, including the reception unit, analysis unit, provision unit, decision unit, monitoring unit, and additional provision unit, is implemented by at least one of, for example, the smart glasses 214 and the data processing apparatus 12. For example, the reception unit receives input from the user via an application of the smart glasses 214. The analysis unit is implemented by a specific processing unit 290 of the data processing apparatus 12 and analyzes the user's symptoms and conditions using generative AI. The provision unit is implemented by the specific processing unit 290 of the data processing apparatus 12 and provides the analyzed information to an emergency call receiving agency or a medical institution. The decision unit is implemented by the specific processing unit 290 of the data processing apparatus 12 and determines deployment of emergency vehicles or a transport route. The monitoring unit is implemented by a control unit 46A of the smart glasses 214 and continuously monitors the user's symptoms and conditions. The additional provision unit is implemented by the specific processing unit 290 of the data processing apparatus 12 and provides additional information when the user's symptoms worsen. The correspondence between each unit and the apparatus or control unit is not limited to the examples described above and various modifications are possible.

### Third Embodiment

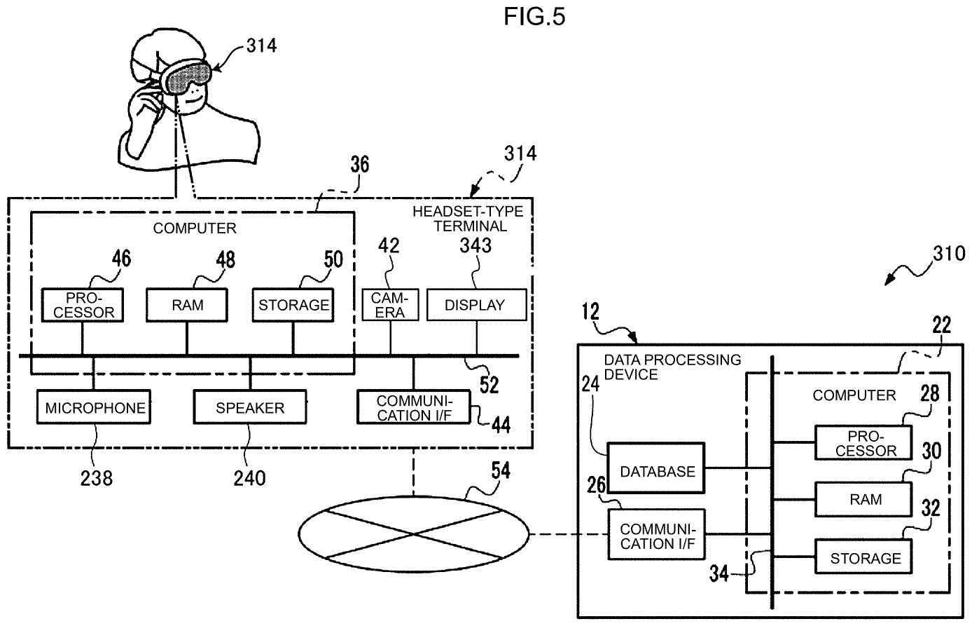

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

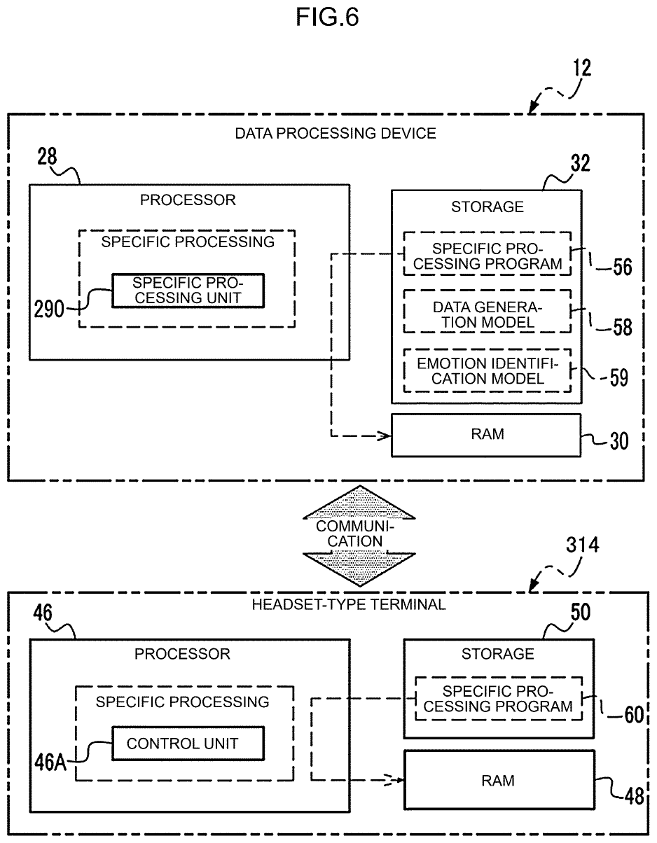

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-mentioned elements, including the reception unit, analysis unit, provision unit, decision unit, monitoring unit, and additional provision unit, is implemented by at least one of, for example, the headset-type terminal 314 and the data processing apparatus 12. For example, the reception unit receives input from the user via an application of the headset-type terminal 314. The analysis unit is implemented by a specific processing unit 290 of the data processing apparatus 12 and analyzes the user's symptoms and conditions using generative AI. The provision unit is implemented by the specific processing unit 290 of the data processing apparatus 12 and provides the analyzed information to an emergency call receiving agency or a medical institution. The decision unit is implemented by the specific processing unit 290 of the data processing apparatus 12 and determines deployment of emergency vehicles or a transport route. The monitoring unit is implemented by a control unit 46A of the headset-type terminal 314 and continuously monitors the user's symptoms and conditions. The additional provision unit is implemented by the specific processing unit 290 of the data processing apparatus 12 and provides additional information when the user's symptoms worsen. The correspondence between each unit and the apparatus or control unit is not limited to the examples described above and various modifications are possible.

### Fourth Embodiment

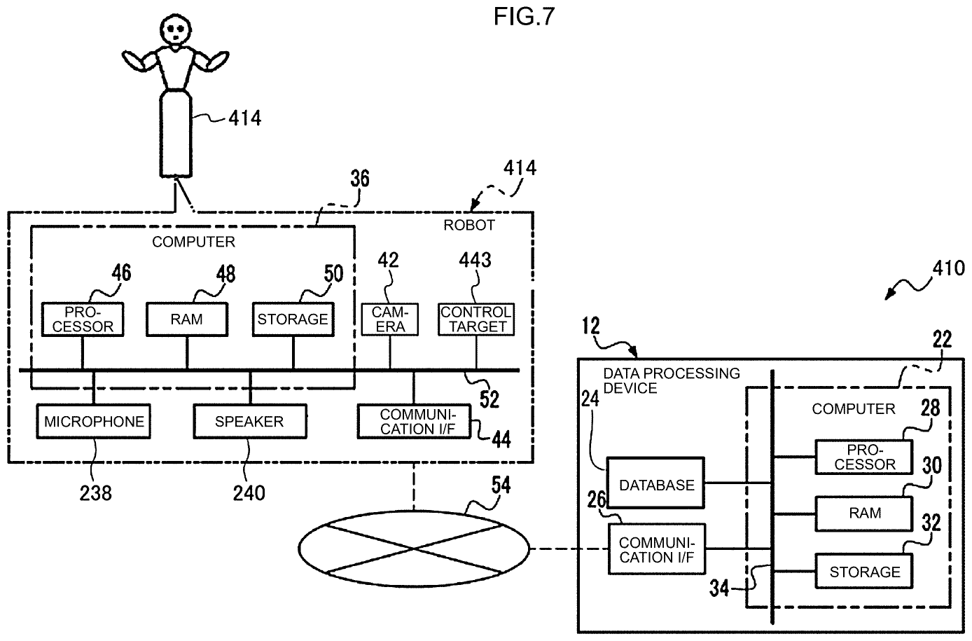

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

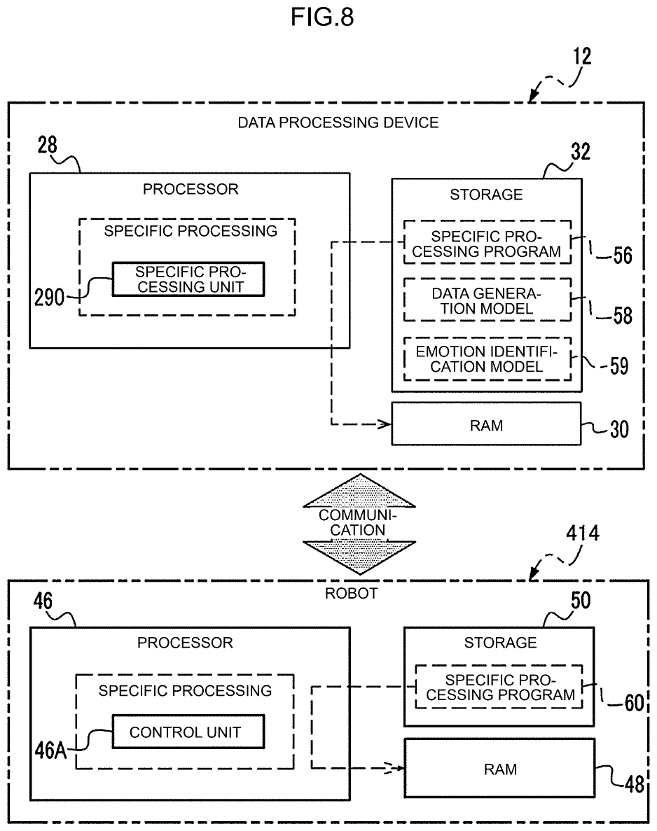

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-mentioned elements, including the reception unit, analysis unit, provision unit, decision unit, monitoring unit, and additional provision unit, is implemented by at least one of, for example, the robot 414 and the data processing apparatus 12. For example, the reception unit receives input from the user via an application of the robot 414. The analysis unit is implemented by a specific processing unit 290 of the data processing apparatus 12 and analyzes the user's symptoms and conditions using generative AI. The provision unit is implemented by the specific processing unit 290 of the data processing apparatus 12 and provides the analyzed information to an emergency call receiving agency or a medical institution. The decision unit is implemented by the specific processing unit 290 of the data processing apparatus 12 and determines deployment of emergency vehicles or a transport route. The monitoring unit is implemented by a control unit 46A of the robot 414 and continuously monitors the user's symptoms and conditions. The additional provision unit is implemented by the specific processing unit 290 of the data processing apparatus 12 and provides additional information when the user's symptoms worsen. The correspondence between each unit and the apparatus or control unit is not limited to the examples described above and various modifications are possible.

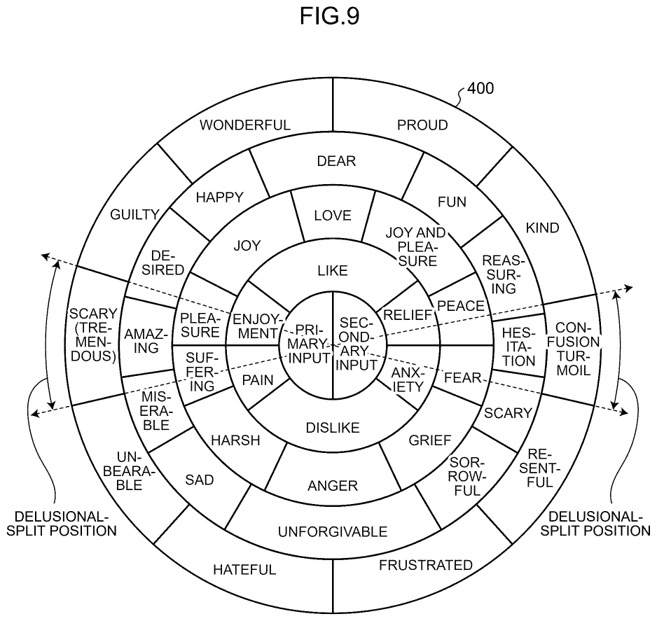

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

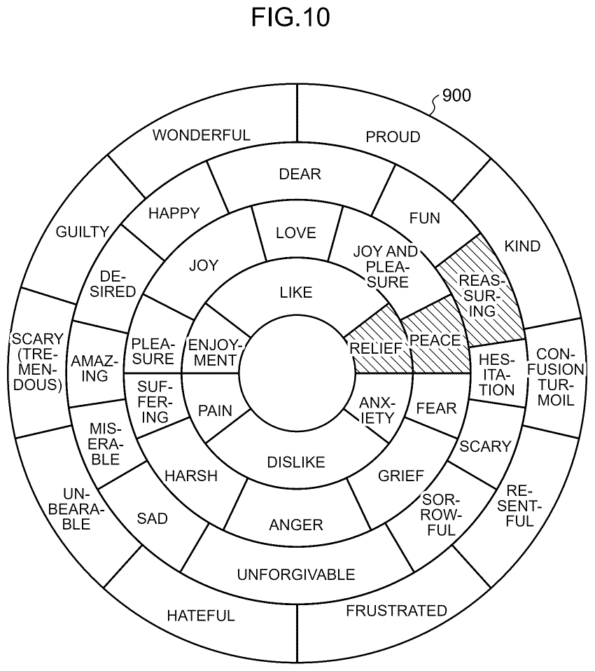

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: a reception unit configured to receive input of a user's symptoms and conditions; an analysis unit configured to analyze information received by the reception unit and determine the user's symptoms and conditions; a provision unit configured to provide information determined by the analysis unit to an emergency call receiving agency or a medical institution; and a decision unit configured to determine deployment of emergency vehicles or a transport route based on the information provided by the provision unit.

(Supplementary Note 2) The system according to Supplementary Note 1, further comprising a monitoring unit configured to continuously monitor the user's symptoms and conditions.

(Supplementary Note 3) The system according to Supplementary Note 2, wherein the monitoring unit comprises an additional provision unit configured to provide additional information when the user's symptoms worsen.

(Supplementary Note 4) The system according to Supplementary Note 1, wherein the analysis unit is configured to analyze the user's symptoms and conditions and arrange appropriate rescue.

(Supplementary Note 5) The system according to Supplementary Note 1, wherein the provision unit is configured to provide detailed information to an emergency call receiving agency or a medical institution.

(Supplementary Note 6) The system according to Supplementary Note 1, wherein the decision unit is configured to determine deployment of emergency vehicles or an optimal transport route.

(Supplementary Note 7) The system according to Supplementary Note 1, wherein the reception unit is configured to estimate the user's emotions and adjust the input method for symptoms and conditions based on the estimated emotions.

(Supplementary Note 8) The system according to Supplementary Note 1, wherein the reception unit is configured to analyze the user's past emergency call history and provide an appropriate input interface.

(Supplementary Note 9) The system according to Supplementary Note 1, wherein the reception unit is configured to filter input content for symptoms and conditions based on the user's current health status and medical history at the time of input.

(Supplementary Note 10) The system according to Supplementary Note 1, wherein the reception unit is configured to estimate the user's emotions and determine the priority of input content based on the estimated emotions.

(Supplementary Note 11) The system according to Supplementary Note 1, wherein the reception unit is configured to prioritize input of highly relevant information based on the user's current geographic location at the time of input of symptoms and conditions.

(Supplementary Note 12) The system according to Supplementary Note 1, wherein the reception unit is configured to analyze the user's social media activity and prompt input of relevant information at the time of input of symptoms and conditions.

(Supplementary Note 13) The system according to Supplementary Note 1, wherein the analysis unit is configured to estimate the user's emotions and adjust the accuracy of analysis based on the estimated emotions.

(Supplementary Note 14) The system according to Supplementary Note 1, wherein the analysis unit is configured to adjust the level of detail of analysis based on the importance of symptoms and conditions during analysis.

(Supplementary Note 15) The system according to Supplementary Note 1, wherein the analysis unit is configured to apply different analysis algorithms according to the category of symptoms and conditions during analysis.

(Supplementary Note 16) The system according to Supplementary Note 1, wherein the analysis unit is configured to estimate the user's emotions and adjust the display method of analysis results based on the estimated emotions.

(Supplementary Note 17) The system according to Supplementary Note 1, wherein the analysis unit is configured to determine the priority of analysis based on the occurrence time of symptoms and conditions during analysis.

(Supplementary Note 18) The system according to Supplementary Note 1, wherein the analysis unit is configured to adjust the order of analysis based on the relevance of symptoms and conditions during analysis.

(Supplementary Note 19) The system according to Supplementary Note 1, wherein the provision unit is configured to estimate the user's emotions and adjust the method of information provision based on the estimated emotions.

(Supplementary Note 20) The system according to Supplementary Note 1, wherein the provision unit is configured to adjust the content of information provision based on the importance of the emergency call receiving agency or medical institution at the time of information provision.

(Supplementary Note 21) The system according to Supplementary Note 1, wherein the provision unit is configured to apply different provision algorithms according to the category of the emergency call receiving agency or medical institution at the time of information provision.

(Supplementary Note 22) The system according to Supplementary Note 1, wherein the provision unit is configured to estimate the user's emotions and determine the priority of information provision based on the estimated emotions.

(Supplementary Note 23) The system according to Supplementary Note 1, wherein the provision unit is configured to adjust the content of information provision based on the geographic location information of the emergency call receiving agency or medical institution at the time of information provision.

(Supplementary Note 24) The system according to Supplementary Note 1, wherein the provision unit is configured to refer to related literature of the emergency call receiving agency or medical institution and adjust the content of information provision at the time of information provision.

(Supplementary Note 25) The system according to Supplementary Note 1, wherein the decision unit is configured to estimate the user's emotions and adjust the method of determining deployment of emergency vehicles or transport routes based on the estimated emotions.

(Supplementary Note 26) The system according to Supplementary Note 1, wherein the decision unit is configured to refer to past deployment history and select an optimal determination method at the time of determining deployment of emergency vehicles or transport routes.

(Supplementary Note 27) The system according to Supplementary Note 1, wherein the decision unit is configured to adjust the determination content based on current traffic conditions at the time of determining deployment of emergency vehicles or transport routes.

(Supplementary Note 28) The system according to Supplementary Note 1, wherein the decision unit is configured to estimate the user's emotions and determine the priority of deployment of emergency vehicles or transport routes based on the estimated emotions.

(Supplementary Note 29) The system according to Supplementary Note 1, wherein the decision unit is configured to select an appropriate determination method based on geographic location information at the time of determining deployment of emergency vehicles or transport routes.

(Supplementary Note 30) The system according to Supplementary Note 1, wherein the decision unit is configured to refer to related literature and adjust the determination content at the time of determining deployment of emergency vehicles or transport routes.

(Supplementary Note 31) The system according to Supplementary Note 2, wherein the monitoring unit is configured to estimate the user's emotions and adjust the frequency of monitoring based on the estimated emotions.

(Supplementary Note 32) The system according to Supplementary Note 2, wherein the monitoring unit is configured to refer to the user's past health data and select an optimal monitoring method during monitoring.

(Supplementary Note 33) The system according to Supplementary Note 2, wherein the monitoring unit is configured to adjust the content of monitoring based on the user's current living conditions during monitoring.

(Supplementary Note 34) The system according to Supplementary Note 2, wherein the monitoring unit is configured to estimate the user's emotions and determine the priority of monitoring based on the estimated emotions.

(Supplementary Note 35) The system according to Supplementary Note 2, wherein the monitoring unit is configured to select an optimal monitoring method in consideration of the user's geographic location information during monitoring.

(Supplementary Note 36) The system according to Supplementary Note 2, wherein the monitoring unit is configured to analyze the user's social media activity and adjust the content of monitoring during monitoring.

(Supplementary Note 37) The system according to Supplementary Note 3, wherein the additional provision unit is configured to estimate the user's emotions and adjust the method of providing additional information based on the estimated emotions.

(Supplementary Note 38) The system according to Supplementary Note 3, wherein the additional provision unit is configured to refer to the user's past health data and select an optimal provision method when providing additional information.

(Supplementary Note 39) The system according to Supplementary Note 3, wherein the additional provision unit is configured to adjust the content of provision based on the user's current living conditions when providing additional information.

(Supplementary Note 40) The system according to Supplementary Note 3, wherein the additional provision unit is configured to estimate the user's emotions and determine the priority of additional information based on the estimated emotions.

(Supplementary Note 41) The system according to Supplementary Note 3, wherein the additional provision unit is configured to select an optimal provision method in consideration of the user's geographic location information when providing additional information.

(Supplementary Note 42) The system according to Supplementary Note 3, wherein the additional provision unit is configured to analyze the user's social media activity and adjust the content of provision when providing additional information.

What is claimed is:

1. A system comprising:
circuitry configured to:
receive, from a client terminal via a packet-switched network, input data comprising at least one of voice data, text data, image data, and time-series sensor data;
preprocess the input data to generate a multidimensional feature tensor by performing at least one of spectrogram conversion on the voice data, vectorization on the text data, feature extraction by a convolutional neural network on the image data, and time-series analysis by a recurrent neural network on the time-series sensor data;
generate, by inputting the multidimensional feature tensor into a Transformer-based multimodal inference model, inference data comprising a severity score and a classification label;
transmit the inference data as structured data to an external server via the packet-switched network; and
generate, based on the inference data and geographic coordinate data, routing data by applying a shortest-path search algorithm in cooperation with a geographic information system, and transmit the routing data to the external server.

2. The system according to claim 1, wherein the input data further comprises vital sign data including at least one of heart rate data, blood pressure data, and blood oxygen saturation data, and the circuitry is further configured to handle the vital sign data as a time-series tensor representing changes over a predetermined time window.

3. The system according to claim 1, wherein the circuitry is further configured to continuously monitor the input data received from the client terminal and generate an anomaly score by applying an anomaly detection model to the time-series sensor data.

4. The system according to claim 3, wherein the anomaly detection model comprises an autoencoder trained to compute a reconstruction error, and the circuitry is further configured to generate an alert flag when the anomaly score exceeds a predetermined threshold.

5. The system according to claim 3, wherein the circuitry is further configured to, in response to the anomaly score exceeding a predetermined threshold, generate additional inference data comprising recent time-series vital data, location coordinate data, and a recommended response label, and transmit the additional inference data to the external server.

6. The system according to claim 1, wherein the circuitry is further configured to estimate an emotion category and an emotion confidence score of a user of the client terminal by inputting at least one of acoustic features extracted from the voice data and expression features extracted from the image data into the Transformer-based multimodal inference model.

7. The system according to claim 6, wherein the circuitry is further configured to dynamically adjust a configuration of a user interface presented on the client terminal based on the emotion category, the adjusting comprising at least one of simplifying input options when the emotion category indicates a high-stress state and expanding input options when the emotion category indicates a low-stress state.

8. The system according to claim 6, wherein the circuitry is further configured to adjust at least one of an analysis threshold, a feature selection parameter, and an analysis depth of the Transformer-based multimodal inference model based on the emotion confidence score.

9. The system according to claim 1, wherein the circuitry is further configured to retrieve, from a history database, past interaction records associated with a user identifier of the client terminal and determine a preferred input modality based on frequency analysis of the past interaction records.

10. The system according to claim 1, wherein the circuitry is further configured to obtain electronic health record data associated with a user of the client terminal and generate a prioritized list of input candidates by inputting the electronic health record data and the input data into a filtering module of the Transformer-based multimodal inference model.

11. The system according to claim 1, wherein the circuitry is further configured to determine, based on the severity score and the classification label, a priority level for the structured data transmitted to the external server.

12. The system according to claim 1, wherein the structured data is formatted according to a medical data interchange standard, and the circuitry is further configured to perform encryption processing and delivery confirmation on the structured data prior to transmission.

13. The system according to claim 1, wherein the circuitry is further configured to obtain real-time traffic condition data via the packet-switched network and recalculate the routing data in response to a change in the traffic condition data.

14. The system according to claim 1, wherein the shortest-path search algorithm comprises at least one of Dijkstra's algorithm and an A* algorithm, and the geographic information system provides map data and road network data for computing the routing data.

15. The system according to claim 1, wherein the circuitry is further configured to retrieve past routing records from a routing database and select an optimal routing strategy based on a success rate computed from the past routing records.

16. The system according to claim 1, wherein the spectrogram conversion comprises mel-frequency cepstral coefficient extraction, and the feature extraction by the convolutional neural network comprises normalizing the image data as an image tensor and extracting a feature vector from the normalized image tensor.

17. The system according to claim 1, wherein the circuitry is further configured to obtain geographic location data from the client terminal and generate a location-based prioritized input list by combining the geographic location data with external data obtained from a geographic information system database.

18. A system comprising:
circuitry configured to:
receive, from a client terminal via a packet-switched network, multimodal input data comprising voice data, text data, image data, and time-series sensor data;
perform spectrogram conversion and mel-frequency cepstral coefficient extraction on the voice data to generate an acoustic feature vector;
perform tokenization and vectorization on the text data to generate a text feature vector;
normalize the image data as an image tensor having dimensions of height, width, and color channels, and extract an image feature vector by applying a convolutional neural network to the image tensor;
analyze the time-series sensor data by a long short-term memory network to generate a time-series feature vector;
concatenate the acoustic feature vector, the text feature vector, the image feature vector, and the time-series feature vector into a multidimensional feature tensor;
generate, by inputting the multidimensional feature tensor into a Transformer-based multimodal inference model, inference data comprising a severity score having a value in a continuous range, a classification label, and a recommended action label;
transmit the inference data as structured data to an external server via the packet-switched network; and
generate, based on the inference data and geographic coordinate data obtained from the client terminal, routing data by applying a shortest-path search algorithm in cooperation with a geographic information system, and transmit the routing data to the external server.

19. The system according to claim 18, wherein the circuitry is further configured to estimate an emotion category by inputting the acoustic feature vector and the image feature vector into the Transformer-based multimodal inference model, and dynamically adjust at least one of an analysis threshold and an analysis depth applied to the multimodal input data based on the emotion category.

20. A method performed by circuitry of a system, the method comprising:
receiving, from a client terminal via a packet-switched network, input data comprising at least one of voice data, text data, image data, and time-series sensor data;
preprocessing the input data to generate a multidimensional feature tensor by performing at least one of spectrogram conversion on the voice data, vectorization on the text data, feature extraction by a convolutional neural network on the image data, and time-series analysis by a recurrent neural network on the time-series sensor data;
generating, by inputting the multidimensional feature tensor into a Transformer-based multimodal inference model, inference data comprising a severity score and a classification label;
transmitting the inference data as structured data to an external server via the packet-switched network; and
generating, based on the inference data and geographic coordinate data, routing data by applying a shortest-path search algorithm in cooperation with a geographic information system, and transmitting the routing data to the external server.
